(19) United States
(12) Patent Application Publication
(10) Pub. No.: US 2026/0368897 A1
(43) Pub. Date: Nov. 19, 2026

(54) AUTONOMOUS DERMOSCOPIC SKIN IMAGING AND LESION RISK ASSESSMENT SYSTEMS AND METHODS

(71) Applicant: SkinBit Inc., Manhattan Beach, CA (US)

(72) Inventor: Jonathan Benassaya, Manhattan Beach, CA (US)

(21) Appl. No.: 19/669,079

(22) Filed: May 6, 2026

Related U.S. Application Data
(60) Provisional application No. 63/805,077, filed on May 13, 2025.
(60) Provisional application No. 63/909,260, filed on Oct. 31, 2025.

(51) Int. Cl. G06T 7/00; A61B 5/00; G06T 7/11; G06T 7/73; G06T 17/00; G06V 10/26; G06V 10/764; G16H 50/20

(52) U.S. Cl. CPC G06T 7/0014; A61B 5/0077; A61B 5/444; A61B 5/7267; A61B 5/7275; G06T 7/11; G06T 7/74; G06T 17/00; G16H 50/20; A61B 2576/02; G06T 2200/04; G06T 2207/10012; G06T 2207/10028; G06T 2207/10148; G06T 2207/20132; G06T 2207/20221; G06T 2207/30088; G06T 2207/30096; G06T 2210/22; G06T 2210/41; G06T 2210/56; G06V 10/267; G06V 10/764; G06V 2201/03; G06V 2201/10

(57) ABSTRACT

Systems and methods for autonomous dermoscopic skin imaging and lesion risk assessment can include a guidance module, high-resolution optical sensors, and a processor coupled to the guidance module and the optical sensors. The processor can: instruct the optical sensors to capture images of two or more positions in regions of skin of a patient using two or more different focal depths; process the images to generate a 3D digital model; segment objects in the 3D digital model using a segmentation AI model; classify the segmented objects into one or more categories using a classification AI module where one of the one or more categories includes skin lesions; assign malignancy risk scores to one or more of the classified segmented objects using the classification AI module; and generate a representation of the 3D digital model with metadata and associated risk scores for the one or more classified segmented objects.

### RELATED APPLICATIONS

This application claims the priority benefit of U.S. Provisional Patent App. No. 63/805,077, filed on May 13, 2025, and titled “AUTONOMOUS DERMOSCOPIC FULL-BODY IMAGING SYSTEM WITH AI-BASED LESION RISK ASSESSMENT AND 3D SKIN RECONSTRUCTION”; and U.S. Provisional Patent App. No. 63/909,260, filed on Oct. 31, 2025, and titled “AUTONOMOUS DERMOSCOPIC SKIN IMAGING AND LESION RISK ASSESSMENT SYSTEMS AND METHODS”, the contents of each of which are incorporated herein by reference in their entirety.

### TECHNICAL FIELD

This disclosure relates generally to the field of dermatology, and more specifically to the field of dermatological skin assessment. Described herein are autonomous dermoscopic skin imaging and lesion risk assessment systems and methods.

### BACKGROUND

The examination of skin lesions and detection of skin cancer are crucial aspects of dermatological practice. Dermatologists currently employ various techniques to examine the skin of patients, including visual inspection, dermoscopy, and photography. Visual inspection involves a manual examination of the skin of a patient by a trained medical professional, who uses their naked eye to identify potential abnormalities. Dermoscopic techniques involve using handheld instruments with magnifying lenses (e.g., a dermatoscope) or cameras to visualize the skin in greater detail. Photography is often used as an adjunctive tool to document and monitor changes in skin lesions over time.

In clinical practice, dermatologists typically focus on examining localized areas of the body, such as the face, using these techniques. These dermoscopic examinations are often performed manually by trained medical professionals, who must carefully examine each area of interest using specialized instruments or cameras. This approach allows them to detect early signs of skin cancer and other conditions.

### SUMMARY

In some aspects, the techniques described herein relate to a system for autonomous dermoscopic skin imaging and lesion risk assessment including: a guidance module including one or more three-dimensional (3D) imaging sensors configured to detect locations of one or more regions of skin of a patient; a plurality of high-resolution optical sensors configured to capture images of the one or more regions of the skin of the patient; and a processor communicatively coupled to a memory, the guidance module, and the plurality of high-resolution optical sensors, wherein the processor is configured to read instructions stored in the memory which cause the processor to: instruct the plurality of high-resolution optical sensors to focus on two or more positions that correspond to the locations of the one or more regions using two or more different focal depths, and capture images while focused on those positions; process the images to generate a 3D digital model of the skin of the patient; segment objects in the 3D digital model using a segmentation AI model; classify the segmented objects into one or more categories using a classification AI module trained on biopsy-confirmed dermoscopic datasets, wherein one of the one or more categories includes skin lesions; assign malignancy risk scores to one or more of the classified segmented objects using the classification AI module; and generate a representation of the 3D digital model with metadata and associated risk scores for the one or more classified segmented objects.

In some aspects, the techniques described herein relate to a computer-implemented method for autonomous dermoscopic skin imaging and lesion risk assessment, including: instructing one or more three-dimensional (3D) imaging sensors to measure locations of one or more regions of skin of a patient; instructing a plurality of high-resolution optical sensors to capture images of the skin, wherein the plurality of high-resolution optical sensors is instructed to focus on two or more positions using two or more different focal depths that correspond to the locations of the one or more regions of the skin of the patient; processing the images to generate a 3D digital model of the skin; segmenting objects in the 3D digital model; classifying the segmented objects into categories using a classification artificial intelligence (AI) module trained on biopsy-confirmed dermoscopic datasets, wherein one of the categories includes skin lesions; assigning malignancy risk scores to one or more of the classified segmented objects using the classification AI module; and generating a representation of the 3D digital model with metadata and associated risk scores for the one or more classified segmented objects.

In some aspects, the techniques described herein relate to a method of controlling an autonomous dermoscopic skin imaging and lesion risk assessment system, including: measuring locations of one or more regions of skin of a patient using one or more three-dimensional (3D) imaging sensors of the autonomous dermoscopic skin imaging and lesion risk assessment system; capturing images of the skin using a plurality of high-resolution optical sensors of the autonomous dermoscopic skin imaging and lesion risk assessment system, wherein the plurality of high-resolution optical sensors is instructed, by a processor, to focus on two or more positions using two or more different focal depths that correspond to the locations of the one or more regions of the skin of the patient; processing the images to generate a 3D digital model of the skin using the processor; segmenting and classifying the one or more regions of the skin as one or more skin lesions in the 3D digital model using an artificial intelligence (AI) module trained on biopsy-confirmed dermoscopic datasets; assigning malignancy risk scores to each identified skin lesion using the AI module; and generating a representation of the 3D digital model with metadata and associated risk scores for one or more skin lesions.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIGS. 1A and 1B show some examples of scanning apparatuses, which include a guidance module, optical sensors (i.e., image sensors), depth sensors, controlled lighting, a processor, and a display.

FIG. 1C shows an example of a scanning apparatus that is similar to the scanning apparatus in FIG. 1A.

FIG. 2A shows a perspective view of an example of a scanning apparatus, which includes optical sensors coupled to a support structure (with the shape of a ring or an ellipse) around a patient.

FIG. 2B shows a zoomed in view of the scanning apparatus of FIG. 2A.

FIG. 2C shows another zoomed in view of the scanning apparatus of FIG. 2A.

FIG. 2D shows a perspective view of an example of a scanning apparatus, which includes an optical sensor assembly coupled to a support structure.

FIG. 2E shows a perspective view of an example of the scanning apparatus of FIG. 2D with a person positioned at least partially within the scanning apparatus.

FIGS. 3A-3C show examples of methods for autonomous dermoscopic skin imaging and lesion risk assessment, which can be executed using an autonomous dermoscopic skin imaging and lesion risk assessment system described herein, such as any of the systems shown in FIGS. 1A-2E.

FIG. 3D shows an example of a computing system, including a processor and computer memory.

FIG. 4A shows an example of a method for dermoscopic skin imaging and lesion risk assessment.

FIG. 4B shows an example of a lesion that is tracked over time using the systems and/or methods described herein.

FIG. 5A shows an example schematic of a portion of a system that can be used to acquire images of a 3D surface using image sensors.

FIGS. 5B-5D show examples of top-down views of three-dimensional (3D) digital models that can be made using any of the systems and methods described herein, for example, any of the systems in FIGS. 1A-2E and 5A.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In clinical practice, dermatologists typically focus on examining localized areas of the body, such as the face, using handheld instruments with magnifying lenses (such as a dermatoscope) or cameras. These examinations are often performed manually by trained medical professionals, who must carefully examine each area of interest using specialized instruments or cameras. This approach allows them to detect early signs of skin cancer and other conditions, but it is labor-intensive, physician dependent, and may not provide a comprehensive view of the patient's overall skin health.

The early detection and diagnosis of skin lesions improve effective treatment and prognosis. Traditional dermoscopy methods rely heavily on human interpretation, which can be subjective and prone to errors. Only some areas of skin are typically observed by a physician using a dermatoscope, and the interpretation of the small selection of observed skin areas is subjective and prone to errors. The autonomous systems described herein are advantageously capable of capturing high-resolution images of human skin and accurately identifying skin lesions to improve the accuracy and efficiency of skin lesion detection. In some cases, the autonomous systems described herein are also advantageously capable of capturing high-resolution images of greater than about 70%, greater than about 80%, greater than about 90% of the skin of the patient, or substantially the whole skin surface of the patient.

The autonomous nature of the systems and methods described herein enable more accurate lesion risk assessments by minimizing the errors associated with human interpretation. The AI-based analysis capabilities can process large amounts of data quickly and accurately, providing a comprehensive assessment of skin lesions with decreased bias and variability compared to conventional approaches. This leads to improved patient outcomes as patients and medical professionals are able to make informed decisions based on reliable diagnostic information provided by the systems and methods described herein. Furthermore, the ability of the systems to reconstruct three-dimensional (3D) images of the skin, rather than the 2D images that are used in conventional dermoscopy, provides valuable insights into lesion morphology and behavior over time, enabling more effective monitoring and treatment planning.

In addition to improved accuracy, the autonomous systems and methods described herein can enable greater accessibility and lower cost than conventional systems and methods. Unlike traditional dermoscopy techniques that require a doctor or skilled technician to capture images of the skin of a patient, the autonomous systems and methods described herein can advantageously increase access to dermatological care and reduce wait times, particularly in underserved areas where specialized medical professionals may not be readily available.

For example, early detection of cutaneous malignancies, including melanoma and the more prevalent non-melanoma skin cancers, basal cell carcinoma (BCC) and squamous cell carcinoma (SCC), materially improves outcomes and reduces treatment complexity, morbidity, and cost. Melanoma prognosis is highly stage dependent. BCC, while rarely metastatic, can cause substantial local tissue destruction requiring extensive excision or reconstruction when detected late. SCC carries a meaningful risk of regional and distant spread with delayed diagnosis. Yet current practice relies on opportunistic spot-checks and clinician-driven dermoscopy that are subjective, vary across operators, and are impractical for comprehensive full body coverage or longitudinal comparison of numerous lesions, particularly in high-risk or heavily sun-damaged patients.

The autonomous systems described herein acquire standardized, high-resolution, dermoscopic-quality images, in some cases across substantially the entire skin surface, and apply repeatable and objective analytics to identify, prioritize, and track suspicious lesions over time. By increasing exam sensitivity and specificity at scale while reducing observer variability and missed anatomic regions, the systems and methods described herein have several advantages including: shortening time-to-evaluation, enabling earlier intervention for skin conditions such as melanoma, BCC, and SCC; improving the outcome for the patients while decreasing the treatment costs for the healthcare system; and decreasing morbidity associated with larger excisions and advanced disease.

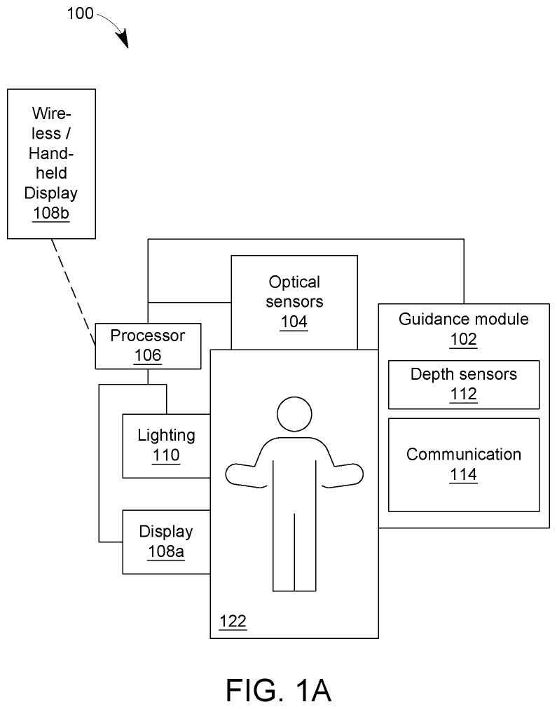

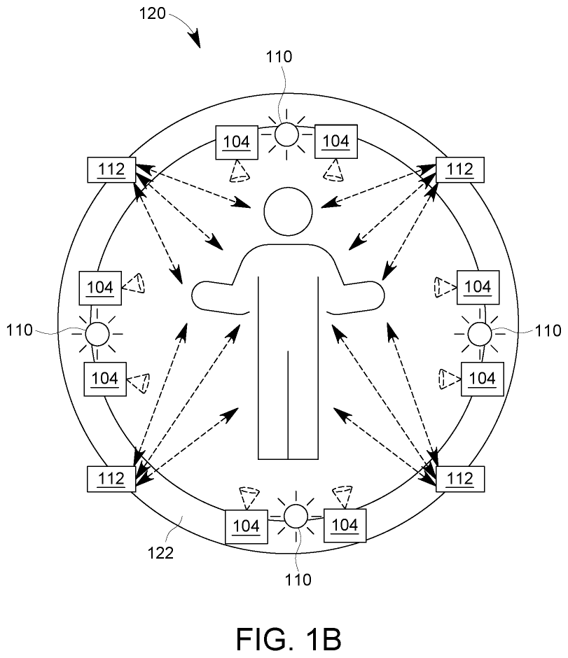

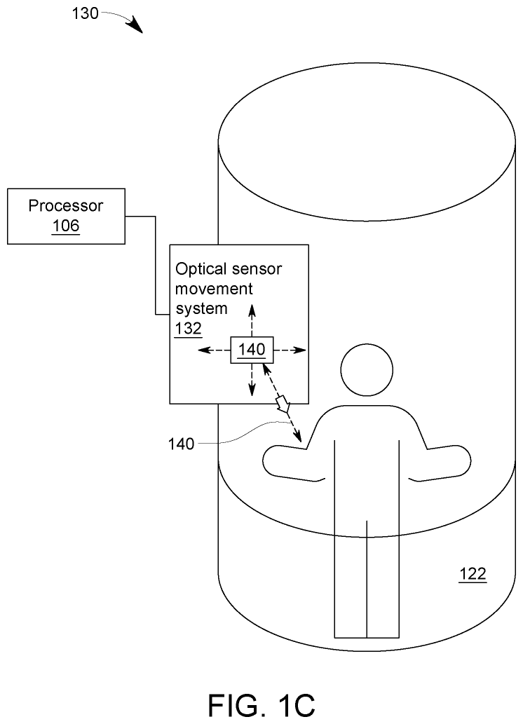

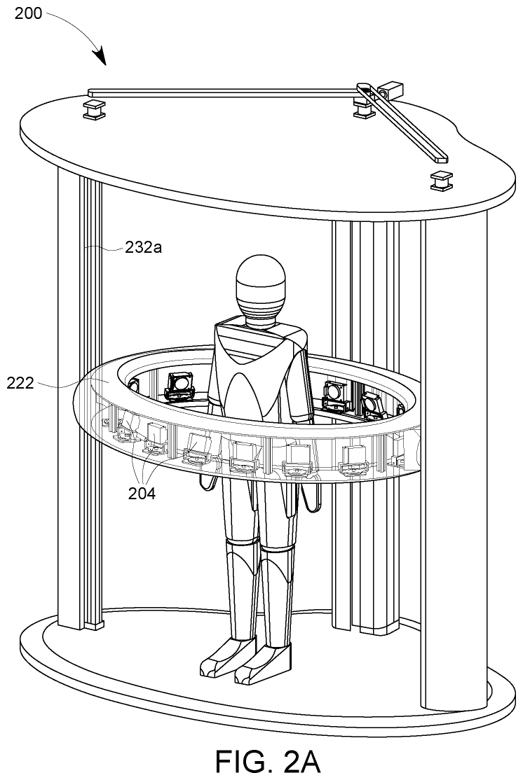

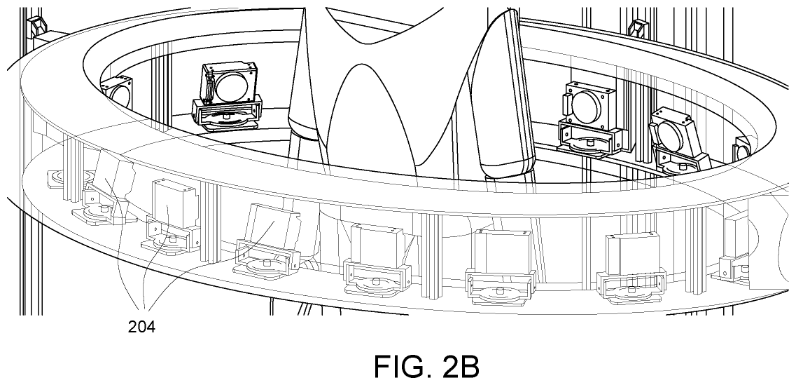

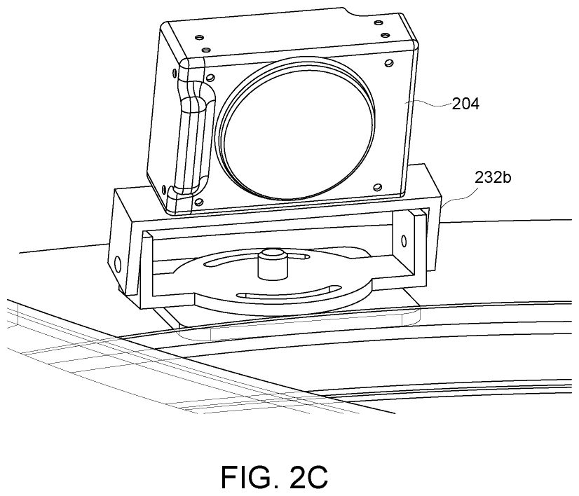

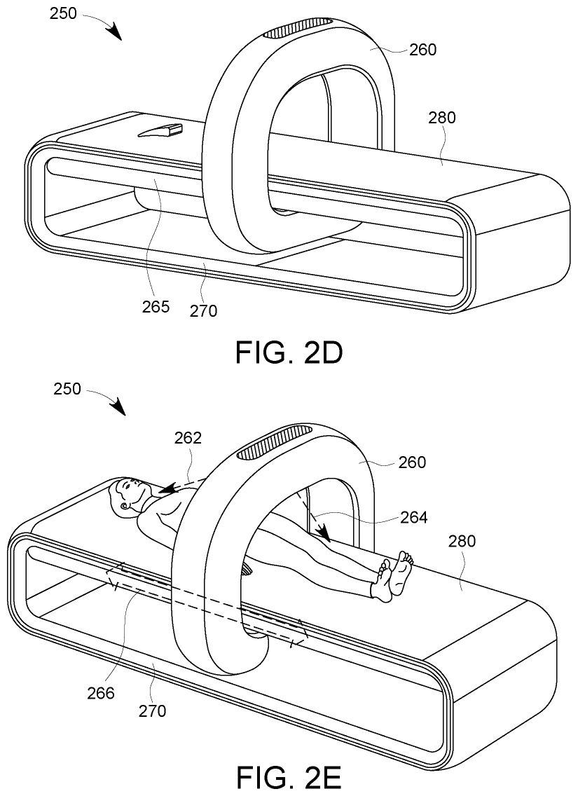

The systems and methods described herein relate to tangible systems (e.g., machine-readable systems) and methods for automated skin health imaging and analysis. These systems and methods are specifically designed to operate on physical devices, such as the scanning apparatuses in FIGS. 1A-2E, which capture and process high-resolution images of the skin of a patient. The hardware and software components of the systems work together to obtain (e.g., through actions of the guidance module, as described herein) and analyze the dermoscopic images to provide lesion risk assessments. As such, the automated skin health imaging and analysis systems and methods herein include specific implementations to solve practical problems in the field, such as availability, reproducibility, and accuracy, which are lacking in conventional solutions for monitoring skin health.

The autonomous skin health imaging and analysis systems and methods described herein offer significant advantages over conventional, human-based dermoscopy approaches.

While trained doctors with dermatoscopes are skilled in interpreting skin lesions, they are inherently limited by their own cognitive biases, fatigue, and the subjective nature of visual interpretation. In contrast, these automated systems and methods can provide more accurate and consistent assessments through the application of repeatable algorithms and machine learning techniques to large datasets. For instance, a doctor may miss subtle changes or patterns on the skin of a patient due to the limitations of human perception, such as difficulties in distinguishing between similar-appearing lesions or overlooking small details that are not immediately apparent. In contrast, these autonomous systems and methods can analyze images at a much higher resolution and with greater objectivity than a human observer, allowing for more accurate detection and diagnosis of skin lesions. Additionally, the automated systems and methods herein can process large volumes of data quickly and efficiently, enabling the identification of patterns and trends that may be difficult or impossible to detect through manual review alone.

The systems and methods described herein solve specific, concrete technical problems that arise in the physical, machine-implemented acquisition and analysis of high-resolution dermoscopic images. Each element of the systems and methods contributes to solving such technical problems in a technically specific and unconventional manner. For example, the guidance module described herein, combines 3D imaging sensors with a processor-driven patient positioning feedback loop and a communication device, solves this technical problem in an unconventional, machine-implemented manner. Specifically, the guidance module detects a three-dimensional position of a patient in real time, compares the position to a reference position stored in memory, and autonomously generates and communicates corrective prompts to the patient until a positional tolerance is satisfied, at which point image acquisition is triggered. This closed-loop, machine-implemented positional feedback mechanism is a specific technical improvement to the functioning of the imaging system itself.

In addition, high-resolution dermoscopic imaging of a curved, three-dimensional biological surface such as human skin presents a specific technical challenge not present in conventional flat-surface imaging. That challenge is that the depth-of-field (DOF) of a high-resolution optical sensor is inherently shallow, such that a single fixed-focus image of a curved skin surface will contain regions that are out of focus and therefore not captured at the intended resolution of approximately 20 microns to 25 microns. The systems and methods described herein solve this technical problem through a specific, unconventional machine-implemented process in which 3D imaging sensors first measure the topology of the skin surface, the processor computes focus vectors corresponding to the measured 3D surface, the high-resolution optical sensors are instructed to capture multiple images at different focus depths corresponding to those vectors, and the processor then applies focus stacking algorithms to combine the in-focus regions of the multiple images into a composite image with high resolution across the entire curved surface. This specific technical sequence, involving the physical interaction between depth sensors, optical sensors, a processor, and the three-dimensional surface of a patient's skin provides a technically specific solution to a technical problem arising from the physical and optical properties of high-resolution imaging systems applied to curved biological surfaces.

The segmentation and classification operations performed by the processor on the 3D digital model also represent specific, unconventional implementations that go beyond generic data analysis. The segmentation AI model used in conjunction with task-specific segmentation models may operate on the high-resolution images and three-dimensional digital models to produce per-pixel probability maps that delineate the boundaries of skin lesions and other skin conditions. The specific technical architecture of this segmentation pipeline, including the combination of foundation models and task-specific models, synthetic image generation to address class imbalance, and the integration of segmentation outputs with the 3D digital model, is not a well-understood, routine, or conventional activity in the field of dermoscopy or in the field of computer vision generally.

The systems and methods described herein are therefore directed to specific technical improvements in the fields of dermoscopic imaging and AI-based skin lesion analysis, implemented through a specific and unconventional combination of physical hardware components including any combination of 3D imaging sensors, high-resolution optical sensors, illumination sources, motorized support structures, and communication devices, together with a specific, unconventional software architecture including any combination of guidance, segmentation, and classification modules. The systems described herein represent a specific, machine-implemented system for performing a method that solves concrete technical problems in the physical acquisition and analysis of dermoscopic images.

The present disclosure relates to systems and methods for automated skin health imaging and analysis and, more specifically, to a dermoscopic full-body imaging platform that captures, processes, and evaluates skin data for skin cancer malignancy risk or other skin related conditions. In some cases, the systems and methods described herein operate autonomously, without requiring technician involvement, to capture, process, and evaluate the skin data. The systems and methods described herein can advantageously provide comprehensive and accurate assessments of skin lesions without requiring human intervention during image acquisition.

A system for autonomous dermoscopic skin imaging and lesion risk assessment can include a scanning apparatus including a guidance module, one or more depth sensors, a plurality of high-resolution optical sensors, illumination sources (e.g., controlled lighting), a processor, and a display. The depth sensors detect a position of a patient, and the processor can be used to provide the information from the depth sensors to the guidance module. The guidance module can have multiple functions. For example, it can direct the positioning of the patient before or during image acquisition using visual and/or audio prompts. The guidance module can also provide instructions to the plurality of high-resolution optical sensors including one or more focus vectors including focus distances and focus directions to image a particular or predefined region of the skin of the patient. The optical sensors can capture images of the human skin using the focus vectors provided from the guidance module.

In the systems and methods described herein, one or more polarization filters can be used to reduce glare by minimizing the amount of reflected light that reaches the detector. The use of polarization filter(s) can be particularly useful when imaging skin surfaces with high reflectivity, such as those containing melanin or other pigments.

In some cases, the systems and methods described herein can include polarizing filters in two locations, between each illumination source and the skin of the patient, and between the skin of the patient and each image sensor. For example, a linearly polarized filter (e.g., a linear polarizer) can be placed between the illumination source and the skin (e.g., it can be coupled to the illumination source), such that polarized light is emitted towards the skin surface. Furthermore, a second linear polarizing filter, which is an analyzer or cross-polarizing filter (e.g., an orthogonal polarizer) can be placed between the skin and the image sensor (e.g., coupled to the image sensor). The polarization axis of the analyzer or cross-polarizing filter is perpendicular to the linearly polarized light from the illumination source. The purpose of the analyzer or cross-polarizing filter is to selectively allow certain polarizations of light to pass through while rejecting others. In this case, the analyzer would be designed to reject linearly polarized light scattered or reflected from the skin surface, thereby reducing glare and improving image quality.

In some cases, linearly polarized light is emitted from the illumination source towards the skin surface using a first polarization filter between the illumination source and the skin of a patient. Some of the polarized light may be scattered or reflected by surface features of the skin (e.g., melanin-containing cells). The scattered or reflected photons, which are still linearly polarized, reach the analyzer filter on the detector. The analyzer filter rejects these linearly polarized photons, minimizing their contribution to the image and reducing glare. The combination of linear polarization at the illumination source and orthogonal polarization (cross-polarization) at the detector, can reduce glare and improve the quality of the captured images.

In some cases, the systems and methods described herein include multiple illumination sources and multiple image sensors. In such cases, all of the illumination sources can have polarization filters (positioned between each of the illumination sources and the skin of the patient) oriented in a first direction (e.g., vertical, or perpendicular to the ground). All of the image sensors can have polarization filters (positioned between the skin of the patient and each of the image sensors) that are oriented in a second direction (e.g., horizontal, or parallel with the ground), where the second direction is perpendicular to the first direction. In some cases, the systems and methods described herein can include polarization filters between the skin and one or more of the image sensors and polarization filters between one or more of the illumination sources and the skin.

In some cases, the systems and methods described herein can include polarization filters between the skin and one or more of the image sensors and no polarization filters between the illumination sources and the skin. In such cases, the transmission axis of the polarization filters can be oriented perpendicular to the surface being imaged. If the skin surface being imaged is curved, then the transmission axis of the polarization filters can be oriented perpendicular to a plane tangential to the curved surface being imaged. Some glare reflected off a surface (e.g., the skin of the patient) can be blocked using a polarizer, even if the incident light is not polarized. For example, light reflecting off horizontal skin surfaces can become polarized horizontally, and a vertical polarization filter between the skin and the image sensor can block the horizontally polarized light while allowing the vertically oriented light to pass.

In some cases, the images of the skin are captured from substantially the entire body surface of the patient. For example, the patient can be in a standing position and the optical sensors can be located surrounding the patient or on more than one side of the patient to capture images from substantially the entire body surface (e.g., greater than about 70% or greater than about 90%) of the patient. In another example, the patient can be supine or prone on a table, and the optical sensors can be located above the patient, and the patient can turn over so that images can be captured from both sides of their body (anterior and posterior sides of the body). In the systems and methods described herein, the optical sensors can be stationary or can move, and/or can include motors or optics to allow them to focus on different regions of the skin of the patient.

In some cases, the automated skin health imaging and analysis systems and methods described herein can: capture a first set of high-resolution images of skin of a patient while the patient is in a first position (e.g., prone, supine, orthostatic, or sitting); instruct the patient to change positions (e.g., move a limb, turn around, sit (if orthostatic or prone or supine), orthostatic (if sitting or prone or supine), prone or supine (if orthostatic or sitting), place one or both arms above their head, or move into another particular position) using the guidance module; detect when they are in a position that can be imaged at high resolution using 3D imaging or depth sensors; and then capture a second set of high-resolution images of the patient while they are in the sitting or lying down position.

The processor can combine multiple images captured by the optical sensors to form a 3D digital model (i.e., 3D model) of the skin of the patient. The 3D model can include high-resolution images of the skin, and information about the positions of different regions of the skin in 3D space. In some cases, the captured images overlap with one another, and overlapping regions can be discarded or combined when analyzing a composite image formed from the individual overlapping images. In some cases, overlapping regions that are not used in the composite image are saved for later reference. For example, each image in the composite image can be restored to include any discarded regions. The discarded regions can be used to create a different composite image or can be used in another operation, for example in a quality control check. In some cases, the images are captured at high resolution. For example, the captured images can have a resolution from about 10 micrometers (i.e., microns) to about 200 microns, from about 20 microns to about 25 microns, or less than about 25 microns. The processor can also analyze the 3D model to identify and segment objects of interest in the captured images. The processor can also analyze the 3D model to classify or categorize the segmented objects of interest. For example, the processor can identify and segment lesions in the images of the 3D model and assign a classification, and optionally a malignancy risk score, to each identified lesion.

In the systems and methods described herein, the depth sensors can measure the position of a patient and transmit the information to the processor. The processor can be used to process the information from the depth sensors to create a 3D map or mesh of the human skin.

For example, the processor can use structure-from-motion, multi-view stereo, or neural surface reconstruction algorithms to convert information from one or more depth sensors to create the 3D map. In some cases, each 3D imaging or depth sensor can determine a distance and direction between the sensor and the skin of the patient, for example, using time-of-flight sensors. The processor can direct the optical sensors to focus on the 3D map or mesh, at depths and directions that correspond to locations of the skin of the patient. The processor can combine images taken at different focus depths together into a composite image with some or all regions in focus. In some cases, whether an image is “in focus” can be defined in relative terms using a chosen sharpness metric. A region can be considered in focus if it meets a predefined criterion, for example, if it exceeds a threshold or is sharper than a corresponding region in a reference image. The reference image can be saved in an archive and its sharpness can be compared to that of a captured image using the processor. The operation of combining multiple images (i.e., two or more images) together into a composite image, where at least one of the multiple images is taken at a different focus depth than another of the multiple images, is referred to as “focus stacking.” The composite image can include images from an optical sensor, one or more optical sensors, or some or all of the plurality of high-resolution optical sensors.

In some cases, the processor can instruct one or more of the high-resolution optical sensors to capture images using more than one focus depth. For example, a high-resolution optical sensor can be instructed to capture images using from about two to about ten different focus depths. In some cases, each high-resolution optical sensor in the system can be instructed to capture images using from about one to about ten different focus depths, or from about two to about ten different focus depths. Combining multiple images from one or more of the high-resolution optical sensors with more than one focus depth can be advantageous to capture high-resolution images of a curved surface.

The processor can use information from the depth sensors, including the position of the patient and the location of each captured image on the skin of the patient, to convert the composite image into a 3D model of the skin of the patient. For example, the processor can instruct each image sensor, or one or more image sensors, to capture one or more images using focus vector(s) determined from a map or mesh of skin of a patient (created using the depth sensors) and combine the captured images taken with different focus depths into the 3D model using the map or mesh.

The processor can perform an array of image analysis functions including localizing and segmenting objects in the composite images or 3D models, identifying whether one or more of the segmented objects are skin lesions, and assigning malignancy risk scores to each of the identified lesions. The display can be used to display the 3D digital model with metadata (e.g., location, size, etc.), any changes, such as size, shape, color, in identified skin lesions or conditions, and associated risk scores for each skin lesion. A computing system with the processor and computer memory can include a segmentation module with an artificial intelligence (AI) model or an ensemble of AI models, that is used to localize and segment skin lesions and skin-conditions in one or more captured composite images (e.g., composite images made from multiple images using focus stacking techniques). The segmentation AI model(s) can be trained on a dataset (e.g., a large dataset containing from 10,000 to 50,000 images, or more than 1000 images, or more than 50,000 images) of annotated skin lesions and skin conditions. The computing system can also include a classification module with an AI model or ensemble of AI models, that is used to classify or identify whether one or more of the localized and segmented objects are skin lesions or skin conditions. The classification AI model can be trained on one or more biopsy-confirmed dermoscopic datasets. The classification module can also be used to assign a malignancy risk score to each identified lesion.

FIGS. 1A-1C show examples of scanning apparatuses 100, 120, 130 which include a guidance module 102, optical sensors 104 (i.e., image sensors), depth sensors 112, controlled lighting 110, processor 106, and a display 108a and/or display 108b. One or more of these components (i.e., the guidance module 102, optical sensors 104, depth sensors 112, processor 106, and a display 108a) can be mounted to a support structure 122 coupled to the floor, a frame, or an enclosure, as described further herein. In some cases, one or more of the components can be mounted to a wall or ceiling of a room. For example, one or more of the components can be mounted to an arm (e.g., the 3D imaging sensors can be mounted to an arm using a camera mount) coupled to a frame or stand supported by the floor.

FIGS. 2A-2E show some examples of scanning apparatus systems 200, 250 including optical sensors to capture images of the skin of a patient who is orthostatic or prone or supine, respectively.

In FIGS. 1A and 1B, the scanning apparatus 100, 120 further includes a support structure 122 (e.g., including an enclosure). For example, the optical sensors 104 can be supported by a side or top of the support structure 122. For example, optical sensors 104 can be coupled to the enclosure using stationary or movable mounts (e.g., as shown in FIG. 2C). The display 108a can be local to the support structure 122, and the display 108b optionally can be a remote device, such as a handheld device (e.g., a smartphone or a tablet), or a computer. In the case of the remote display 108b, the processor can communicate wirelessly (e.g., using Wi-Fi, Bluetooth, Zigbee, etc.), for example through the cloud.

The guidance module 102 in FIG. 1A includes, or is in communication with, the depth sensors 112, which can detect the location of a patient in the support structure 122 and transmit information to the processor 106. For example, the depth sensors 112 can be stereoscopic cameras, Time-of-Flight (ToF) sensors, or other laser-based distance measurement sensors (e.g., those using phase shift), that can detect the locations of objects within a field of view. The processor can convert the information from the depth sensors 112 (e.g., using ToF sensors, structure-from-motion, multi-view stereo, or neural surface reconstruction algorithms) into a 3D map of the skin of the patient. The processor 106 can then direct the optical sensors 104 to focus on regions of the patient's skin based on the 3D map, and images from each image sensor, focused on different depths, can be combined into one or more 3D models. In some cases, the 3D models cover greater than about 70%, greater than about 80%, greater than about 90%, or substantially the whole body of the patient. The processor 106 can use information from the depth sensors 112 to guide the optical sensors 104 to focus at the depths and directions (i.e., focus vectors) provided by the depth sensors 112 to capture high-resolution images of the skin of the patient.

The guidance module 102 can also direct positioning of a patient before or during image acquisition using visual and/or audio prompts. For example, if depth sensors 112 detect a position of a patient that is out of range, such that the image sensors 104 cannot focus on the skin to obtain a high-resolution image, the processor 106 can direct the guidance module 102 to send one or more visual and/or auditory communications to the patient instructing them to change their positioning. The guidance module can thereby advantageously enable system 100, 120 to autonomously capture high-resolution images of the skin of the patient without needing a doctor or trained technician to operate the system.

The optical sensors 104 may be high-resolution optical sensors that can capture images of human skin (e.g., at a resolution from about 10 microns to about 200 microns, or from about 20 microns to about 25 microns, or less than about 25 microns). In some cases, there can be from about 2 to about 100 optical sensors, from about 10 to about 100, or more than 100 optical sensors. In some cases, the optical sensors can be mounted on one side of a patient, on multiple sides of the patient, or surrounding the patient (e.g., as shown schematically in FIG. 1B, and in FIG. 2A below).

FIG. 1B shows an example including eight image sensors 104, four depth sensors 112, and four illumination sources 110. In other examples, the systems and methods described herein, such as those shown in FIGS. 1A-2E, can include from about 5 to about 200 image sensors, from about 5 to about 50 depth sensors, and from about 2 to about 1000 illumination sources. The dotted cones for the optical sensors 104 shown in FIG. 1B indicate directions or fields of view for each of the optical sensors and represent directions from which images can be taken using the optical sensors. The optical sensors can be positioned such that their combined fields of view can capture images from greater than about 70%, greater than about 80%, greater than about 90%, or substantially the whole body of the patient. In some cases, the optical sensors can move in order to capture images, and/or can contain optics to allow them to focus on different regions of skin, as described further herein.

The image sensors 104 can be positioned surrounding the patient, on multiple sides of the patient, primarily on one side of the patient, or on only one side of the patient. Some examples are shown in FIGS. 2A-2E. In other cases, the image sensors can be in other geometries and the other aspects of the systems and methods described herein can still be used. For example, the patient can lie down on a patient support apparatus (e.g., table, bed, etc.), and the depth sensors 112 and the image sensors 104 can be mounted all on one side that is above the table (e.g., as shown in FIGS. 2D-2E below). In such cases, the guidance module 102 could instruct the patient to turn over after images were acquired of a first side of their body so that images can be acquired of the second side of their body.

In some cases of the systems and methods described herein, the image sensors (e.g., image sensors 104 in FIGS. 1A-1C) are mounted on a motorized structure (e.g., optical sensor movement system 132 in FIG. 1C) that can move the image sensors to image the skin of the patient. The optical sensor movement system 132 may include motors, rails, hydraulics, linear actuators, gimbal mounts, or other mechanical systems. For example, a system described herein can include a ring of image sensors that can translate in a first direction (e.g., to perform a longitudinal scan of a patient), and each image sensor can be translated toward the patient in a second direction (e.g., where the second direction is approximately perpendicular to the first direction) to increase the resolution. A processor (e.g., processor 106) can instruct the motorized structure where to move the image sensors, and in which direction to orient them. The processor can instruct the system to image a first region of skin of the patient using the image sensors, then move the image sensors using the motorized structure, and image a second region of skin of the patient. In some cases, a patient can be lying down (e.g., on a table or bed), and the motorized structure can move an image sensor to image different regions of skin of the patient.

In some cases, there can be one motorized structure to move one image sensor or a set of image sensors to image the body of the patient. In other cases, there can be more than one motorized structure (e.g., two, three, four, from two to ten, or from two to 100), and the processor can instruct each motorized structure to move an image sensor or set of image sensors to image the body of the patient. In some cases, the processor can also instruct the motorized structure(s) to move the image sensor(s) such that they are at a correct distance from the patient. For example, there can be one or more depth sensors (e.g., depth sensors 112 in FIGS. 1A-1C) which can determine the location of the patient, and the information from the depth sensors can be used by the processor to determine instructions for moving and orienting the image sensor(s) using the motorized system(s).

The processor 106 is electrically coupled to the optical sensors 104 and can be used to process the images, for example to create a 3D digital model of the human skin. For example, multiple images taken at different focal depths, and images from multiple image sensors can be combined into one or more composite images, and the composite image can be combined with information from the depth sensors to create the 3D model. In some cases, the depth sensor(s) provide depth images to processor 106, which are de-projected by processor 106 to make 3D point clouds using the optical parameters, for example focal lengths and principal points, of the image sensors. The principal point of an image sensor is a pixel location on the image sensor where the optical axis intersects an image plane of the image sensor. The point clouds can then be processed to generate a 3D surface (i.e., the 3D model). In some cases, the point clouds can be generated by triangulating and/or interpolating information from multiple depth sensors to form a continuous 3D surface representation. The processor 106 can then paint the 3D model using the images from the image sensors, for example, in a process called projection mapping or depth-image fusion. In projection mapping or depth-image fusion, data from the depth sensors (such as ToF sensors, or stereo cameras) and known positions and orientations of the image sensors are used to position images captured by image sensors in 3D space to generate a 3D reconstruction of an object or scene, in this case, a 3D model of the skin of the patient. This technique allows for accurate measurement and representation of both shape and appearance, enabling accurate 3D modeling, tracking, and rendering of objects (e.g., skin lesions) in the images. In some cases, the focal lengths of the image sensors can also be used to position images captured by image sensors in 3D space to generate the 3D model. In some cases, focus stacking is used (where an image sensor captures images at different focal lengths) and images captured using different focal lengths (from one or more image sensors) are positioned in 3D space to generate the 3D model based on information from the depth sensors (e.g., based on one or more 3D point clouds).

Processor 106 can also be used to localize and segment objects in the 3D digital model using one or more segmentation AI models, for example, those leveraging foundation computer-vision models (e.g., SAM 2, DINOv2) alongside a task-specific segmentation (e.g., YOLOv11). In some cases, the segmentation AI models can detect the location (e.g., left, right, arm, leg, torso, etc.) of an image automatically based on computer vision. In some cases, the location of the image sensor that took the image of the skin of the patient and information from the depth sensors can be used to determine the location of an object (e.g., a lesion or skin condition) in an image. The size of the lesion or skin condition in the segmented image can be calculated based on the size in the segmented image (e.g., in total number of pixels), the 3D mesh generated by the depth sensors, and trigonometry.

The segmentation AI models, e.g., foundation computer-vision models, of the systems and methods described herein can automatically identify and segment objects in images, in some cases, without task-specific training. In some cases, the AI models used for segmentation can be trained on large-scale, unlabeled image data sets in order to produce high-quality, general-purpose image embeddings. The embeddings can be used for classification, retrieval, or segmentation, of features in images. In some cases, these foundation computer-vision AI models can outperform supervised AI models, and advantageously can require little or no fine-tuning. In some cases, task-specific segmentation models can detect objects in images and produce pixel-level segmentation masks for each object detected. A task-specific segmentation model can be designed for speed and accuracy, for example, with different segmentation model sizes chosen to balance performance. This can be advantageous for fast or real-time image analysis. In contrast to the foundation models, the task-specific segmentation models are trained for specific detection and segmentation applications rather than broad, general-purpose visual understanding. For example, the task-specific segmentation models described herein can be trained using dermoscopic datasets (e.g., biopsy-confirmed dermoscopic datasets).

Processor 106 can also be used to identify whether one or more of the segmented objects are skin lesions using a classification AI module trained on biopsy-confirmed dermoscopic datasets. A classification AI module can be an ensemble of multiple image classifiers-for example, a mix of EfficientNet-V2 and EVAv2-based transformers, trained with slightly different combinations of widths, depths, resolutions, hyperparameters, and/or random seeds. Ensembling can advantageously reduce error variance and can yield more stable, better-calibrated predictions.

The image classification models of the systems and methods described herein can take an input image and assign it to one or more predefined categories, and can thereby be used to determine what features or objects are present in the image. They can learn patterns such as shapes, colors, and textures from large datasets, and then use those learned features to recognize and label new images, with high accuracy. The image classification models can use optimized scaling methods to balance network depth, width, and resolution, so that they are both accurate and computationally efficient. Large-scale vision transformer models can process images by breaking them into patches and analyzing the relationships between those patches, for example using attention mechanisms. These models analyze images similar to the way language transformers handle words in a sentence. They can be trained on large datasets to learn general-purpose visual representations that can then be adapted to many tasks such as classification, segmentation, and detection. Large-scale vision transformer models can be trained using annotated or labeled datasets (supervised learning) or with unlabeled datasets using self-supervised or weakly supervised methods. For example, contrastive learning or image-text pairing (e.g., using CLIP, DINO, or EVAv2) can be used to train transformer models using large collections of unlabeled or loosely labeled images, thereby reducing the dependence on costly human annotation.

In some cases, the classification models of the systems and methods described herein can further be used to determine a risk score for an object identified in the image. For example, malignancy risk score (e.g., from 0 to 1, from 0% to 100%, or SE[0,1]) can denote the probability that a lesion is malignant (i.e., that it belongs to a malignant class such as melanoma, basal cell carcinoma (BCC), squamous cell carcinoma (SCC), etc.) as opposed to a benign lesion. A risk score can also be made based on a patient-level “atypicality” score. For example, a lesion that has unusual features compared to other objects detected in images of their skin could have a relatively high atypicality score. The atypicality score can be used to determine a risk score, for example, where a higher atypicality score would correspond to a higher risk score.

For example, an atypicality score can be derived from cosine distances between the visual embedding of the lesion and the distribution of embeddings of other lesions of that patient (e.g., embeddings can come from the penultimate layer of a classifier or a model trained with contrastive learning), and morphology metrics from segmentation (e.g., diameter, asymmetry, border irregularity, and/or color variability) combined via a lightweight logistic or gradient-boosting head. Using cosine distance techniques can make the linear/logistic head behave like simpler classifier models, which can advantageously make it lightweight and faster. The lightweight logistic or gradient-boosting head can be a fast, efficient, and small classification layer (e.g., using LightGBM, which is a histogram-based gradient-boosting decision tree framework) trained on top of high-level features extracted by a convolutional base. The use of such a classification layer can provide high accuracy for large datasets while reducing computational overhead.

In other words, an atypicality score can be determined by measuring how different two images (e.g., segmented images) are in the learned feature space of the AI model, based on the distance or angle between their embedding vectors. The phrase “embedding vectors” refers to numerical representations generated by an AI model that capture the attributes of an image within a multi-dimensional space. Images with similar attributes can be represented by vectors that are closer together in the multi-dimensional space. Embedding vectors can therefore be used to compare images to one another and can provide a measure of similarity or difference between images.

Another way to compute the risk score or atypicality score is to derive it from lesion-specific features, such as boundary shape or irregularity, size, and longitudinal change, and to quantify the deviation of these features from the expected distribution for the patient or for a reference population. The atypicality score can also be based on temporal change. For example, a variation in the visual embedding (as produced by the methods described above) of a lesion, or shifts in simple appearance attributes (e.g., size, color) of the lesion, can be measured as absolute or relative differences over time (e.g., area, diameter, color indexes) to determine the atypicality score.

In some cases, the systems and methods described herein can compute several malignancy risk scores and/or atypicality scores using different methods, which are complementary to each other. In some cases, an overall score can be determined using several scores obtained using different input information and/or different AI models. The several scores can all be given equal weights to determine the overall score, or different weights can be used when converting the different scores to the overall score.

The lesions can be localized by saving or reporting the location of the detected lesion in the context of the original image. For example, a segmented outline of the detected lesion can be overlaid on top of the original image at the location of the detected lesion.

In some cases, processor 106 can be located remotely (e.g., in the cloud), and the scanning apparatus 100 can have a local processor and system to communicate with the remote processor 106.

The display 108a and/or display 108b can be coupled to, or in communication with, processor 106 and can be used to display the 3D digital model with metadata (e.g., location, size, etc.) and associated risk scores for one or more skin lesions.

Guidance module 102 in FIG. 1A provides an automated and accurate positioning system for patients before or during image acquisition. The guidance module 102 includes one or more three-dimensional (3D) depth sensors 112, such as stereoscopic cameras, ToF sensors, or other laser-based distance measurement sensors, to detect the positioning of a patient. Information from the depth sensors 112 enables the processor 106 to detect the position of a patient in three dimensions (e.g., using ToF sensor information, structure-from-motion, multi-view stereo, or neural surface reconstruction algorithms), allowing for precise alignment with the scanning apparatus. The guidance module 102 also includes communication device 114 to communicate visual and/or audio prompts to the patient through a visual display or one or more audio speakers, respectively. For example, the guidance module 102 could detect that an arm of a patient is too low (i.e., too close to the ground) and communicate a prompt to the patient to raise their arm. Other examples of prompts that can be communicated from the communication device 114 to the patient are “raise your leg,” “bend your knee to show your foot sole,” “spread your arms wider,” or “rotate by 45 degrees.” The prompts can be either quantitative (e.g., raise your arm by 2 inches) or qualitative (e.g., raise your arm a little bit).

Processor 106 is coupled to the guidance module 102 and can be used to control the depth sensors 112 and communication device 114. The processor 106 can instruct the depth sensors 112 to detect the position of a patient, analyze the detected position by comparing it to a reference position, and direct the communication device 114 to provide visual and/or audio prompts if necessary. For example, the reference position can be a preprogrammed ideal position for measurement, or the reference position can be a position of a specific patient detected in a previous measurement. The ideal position can be a position where all of the optical sensors can focus on the skin of the patient to capture high-resolution images. The reference position can be a sitting position, a standing position, or a position where a patient is lying down (e.g., on an examination table). The processor can instruct the scanning apparatus 100 to proceed with capturing overlapping images of human skin once the detected position matches the reference position within a tolerance. For example, the tolerance can be a fixed length (e.g., 10 cm), or a percentage error (e.g., within 10%) of the distance between the image sensor and the skin.

In some cases, the guidance module 102 includes a processor that can process information from the depth sensors 112 and transmit signals to the communication system 114, and optionally to optical sensors 104. In some cases, the processor of the guidance module 102 can communicate with processor 106, and the processor 106 can process information from the depth sensors 112 and transmit signals to the communication system 114 and optical sensors 104. In other cases, the guidance module 102 does not have a dedicated processor, and processor 106 is coupled to the depth sensors 112 and communication system 114 to transmit and receive signals and control the components of the guidance module 102 as well as other components of the scanning apparatus 100.

The illumination sources 110 (“lighting”) in FIGS. 1A and 1B can be positioned to illuminate the area of skin that the optical sensor may capture. In some cases, each image sensor 104 has one or more illumination sources 110, such that each area that is captured is illuminated by the one or more illumination sources. There can also be divergence of the light such that an illumination source 110 illuminates the capture area from more than one image sensor 104. In some cases, the illumination sources 110 are positioned to avoid glare for the image sensors 104. For example, the angles between the image sensor 104 focus vectors, the skin, and the illumination sources 110 can be chosen to reduce glare. For example, an image sensor 104 focus vector can be roughly normal to a region of skin, and illumination source(s) 110 can be positioned to illuminate the region of skin from an angle that is oblique (e.g., 30°, 45°, or 60° from normal). In some cases, illumination sources 110 illuminate the skin using diffuse light (e.g., by incorporating diffuser panels, louvers, or other diffuser means, with each illumination source 110) to prevent glare.

The illumination sources 110 can include any sources of light, such as halogen lamps, or light emitting diodes (LEDs). In some cases, the illumination sources 110 provide high-intensity, broad-spectrum illumination (e.g., bright white light), for example, to render skin color and texture accurately. For example, high-intensity broad-spectrum light (e.g., high color rendering index (CRI) LEDs or tungsten-halogen sources) can enable accurate digital imaging by providing a balanced spectrum of wavelengths so that the colors in the sample are rendered accurately (e.g., without saturation loss). High intensity illumination (e.g., greater than 10,000 lux, or from 10,000 lux to 450,000 lux per illumination source 110) enables the image sensors to reduce digital noise, and produce high-resolution images. In some cases, illumination sources 110 can illuminate the skin of the patient with polarized light. In some cases, specific wavelengths of light can be used for specific purposes. For example, fluorescent “Wood's lamps” which include UV-A light (about 365 nm) can be used to highlight pigmentation disorders, bacterial or fungal infections, and other skin conditions that fluoresce under UV-A light. For example, image sensors that can detect UV light (e.g., with wavelengths from about 200 nm to about 400 nm) or black light, can be used to capture images of the skin including information related to the UV damage of the skin.

In some cases, illumination sources 110 each include a polarization filter. The polarization filters can be coupled to each illumination source 110. For example, the polarization filter can be attached to the front of each illumination source 110 such that the light emitted by each illumination source 110 passes through a polarization filter. In such cases, linearly polarized light is emitted from the illumination sources 110 towards the skin of the patient.

In some cases, the optical sensors 104 each include a polarization filter. The polarization filters can be coupled to each optical sensor 104. For example, the polarization filter can be attached to the front of each optical sensor 104 such that the light detected by each optical sensor 104 passes through a polarization filter before being detected.

In some cases, illumination sources 110 each include a polarization filter and the optical sensors 104 each include a polarization filter that is an analyzer or cross-polarizing filter. In some cases, all of the polarization filters on the illumination sources 110 have polarization axes that are parallel with one another. In such cases, the polarization axis of each analyzer or cross-polarizing filter can be oriented perpendicular to the polarization axis of the filters on the illumination sources 110, such that they are also perpendicular to the linearly polarized light from the illumination source that reflects off of the skin. For example, illumination sources 110 can each include a polarization filter with a polarization axis oriented horizontally, and the optical sensors 104 can each include an analyzer or cross-polarizing filter with a polarization axis oriented vertically. In some cases, one or more of illumination sources 110 include a polarization filter while other illumination sources 110 do not, and one or more of the optical sensors 104 include a polarization filter that is an analyzer or cross-polarizing filter while other optical sensors 104 do not.

In some cases, illumination sources 110 do not include a polarization filter and one or more, or all, of the optical sensors 104 include a polarization filter that is oriented perpendicular to the surface being imaged, or to a plane tangential to the surface being imaged (if the surface is curved). In such cases, the polarization axis of each polarization filter is perpendicular to some of the linearly polarized light reflecting from the skin to reduce the glare. For example, the polarization axis of each polarization filter of the optical sensors 104 can be oriented vertically with respect to the ground.

In another example, image sensors that detect IR light (e.g., with wavelengths from about 1 micron to about 10 microns, or from 1 micron to about 20 microns) can be used to capture images that include information about the temperature distribution of the skin of a patient. For example, not to be limited by theory, malignant skin lesions can have higher temperatures than surrounding skin due to increased metabolic activity and blood supply. In other cases, some malignant lesions (e.g., with reduced metabolic activity or a poorly developed blood supply) and certain benign lesions (e.g., cysts) may have lower temperatures than surrounding skin. In some cases, an object in the 3D model of the skin of a patient can be classified using a difference in temperature compared to the surrounding areas of skin (either higher or lower in temperature).

FIG. 1C shows an example of a scanning apparatus 130 that is similar to the scanning apparatus 100 in FIG. 1A. The scanning apparatus 130 further includes an optical sensor movement system 132 that can be used to move the optical sensor 104 to a particular distance 140 from the skin. This can be advantageous, for example, to move the optical sensor 104 so that it can focus on the skin of the patient in order to capture high-resolution images. The optical sensor movement system 132 may include any type of movement system including, motors, rails, hydraulics, linear actuators, gimbal mounts, or other mechanical systems. These systems can be coupled to and controlled by the processor 106. The optical sensor movement system 132 can be physically coupled to and supported by the support structure 122. FIGS. 2A-2E show some examples of support structures, and components, including optical sensor movement systems, coupled to them. In other cases, the optical sensor movement system 132 can be supported by another structure, such as a wall or ceiling adjacent to the support structure 122, which can support other components of the system.

FIGS. 2A-2C show an example of a scanning apparatus 200, which includes optical sensors 204 coupled to a support structure 222 (in the shape of a ring or an ellipse, in this case) around a patient. In some cases, the ring of optical sensors 204 is stationary. In other cases, the ring of optical sensors 204 is coupled to optical sensor movement system 232a (e.g., including one or more motors), which can move the ring of optical sensors 204 vertically. In this example, the patient is orthostatic, and the optical sensors 204 can be located surrounding the patient, as shown.

FIGS. 2B and 2C show that each optical sensor 204 is also coupled to a mounting bracket 232b. In some cases, mounting bracket 232b can be a movement system, for example, including a hinge, a horizontal actuator, and a gimbal mounted to the ring. A movement system in the form of mounting bracket 232b can be used to swivel and change the tilt angle of each optical sensor 204, to image a region of the skin of the patient. For example, a processor (e.g., processor 106 in FIGS. 1A-1C) can instruct the optical sensors 204 to aim and focus on a region of skin of the patient based on information from the depth sensors (e.g., depth sensors 112 in FIGS. 1A-1C). In other cases, the optical sensors 204 can be stationary and can focus on different locations (e.g., as instructed by the processor based on depth sensor information) to image a portion of the skin of a patient. In some cases, each of the optical sensors 204 can move horizontally (i.e., in the plane of the ring) using the horizontal actuator of the mounting bracket 232b to get closer to or farther from the patient. The horizontal movement can be beneficial, for example, to move the cameras to a distance from the skin so that they can focus on the skin and obtain high-resolution images (e.g., from about 10 microns to about 200 microns, or from about 20 microns to about 25 microns, or less than about 25 microns). In some cases, all of the optical sensors 204 can capture substantially the whole skin surface of the patient (e.g., greater than about 70%, or greater than about 90% of the skin of a patient) and capture high-resolution images.

FIGS. 2D and 2E show an example of a scanning apparatus 250, which includes optical sensor assembly 260 coupled to a support structure 270, coupled to a table or bed 280, upon which a patient can lie down (e.g., supine, prone, or side). The optical sensor assembly 260 can include the optical sensors (e.g., 104 in FIGS. 1A-1C). The optical sensor assembly 260 can also optionally include the 3D imaging or depth sensors (e.g., 112 in FIGS. 1A-1B), and the lighting 110 (in FIGS. 1A-1B). In some cases, the 3D imaging or depth sensors (e.g., 112 in FIGS. 1A-1B), and the lighting 110 (in FIGS. 1A-1B) can be mounted to another structure (e.g., a wall, ceiling, or another structure of the scanning apparatus) (not shown).

In some cases, the optical sensor assembly 260 is stationary and the optical sensors (not shown) are coupled to it and aimed such that optical sensor assembly 260 can capture high-resolution images of substantially the whole skin surface of the patient (e.g., greater than about 70% or greater than about 90% of the skin of the patient). For example, some of the optical sensors of the optical sensor assembly 260 can be aimed towards the head or face of the patient (e.g., as shown by focus vectors 262) and some of the optical sensors can be aimed at the legs or feet of the patient (e.g., as shown by focus vector 264).

In other cases, the optical sensors assembly 260 is coupled to optical sensor movement system 265 (e.g., including one or more motors), which can move the optical sensors assembly 260 horizontally to image the patient (e.g., as shown by double arrow 266). For example, one or more images can be taken near the head or face of the patient, the optical sensor movement system 265 can move the optical sensors assembly 260 in horizontal direction 266, and one or more images can be taken near the mid-section of the patient. The optical sensor movement system 265 can also move the optical sensors assembly 260 in horizontal direction 266 towards the feet or legs of the patient, and one or more images can be taken near the feet or legs of the patient.

In some cases, the guidance module (e.g., guidance module 102 in FIGS. 1A-1C) can instruct the patient to change positions so that images can be taken of additional regions of the skin of the patient. For example, a set of images can be taken in a first position, and then the patient can be instructed to change positions to a second position, and a second set of images can be taken. For example, in the first position the patient can be supine, and in the second position the patient can be prone. In some cases, the guidance module can instruct the patient to change positions more than once to capture high-resolution images of substantially the whole skin surface of the patient (e.g., greater than about 70% or greater than about 90% of the skin of the patient). For example, the patient can be instructed to be supine, to lie on their left side, to be prone, and/or lie on their right side, in order to capture images. The patient can also be instructed to move one or more of their limbs, for example, to raise their arms over their head, or to bend one of their knees and place their foot on the table 280. The guidance module can also instruct the patient to move into regions where high-resolution images of their skin can be captured, based on information from 3D imaging or depth sensors (e.g., 112 in FIGS. 1A-1B), for example, which can be mounted to the optical sensor assembly 260 or to another structure (not shown).

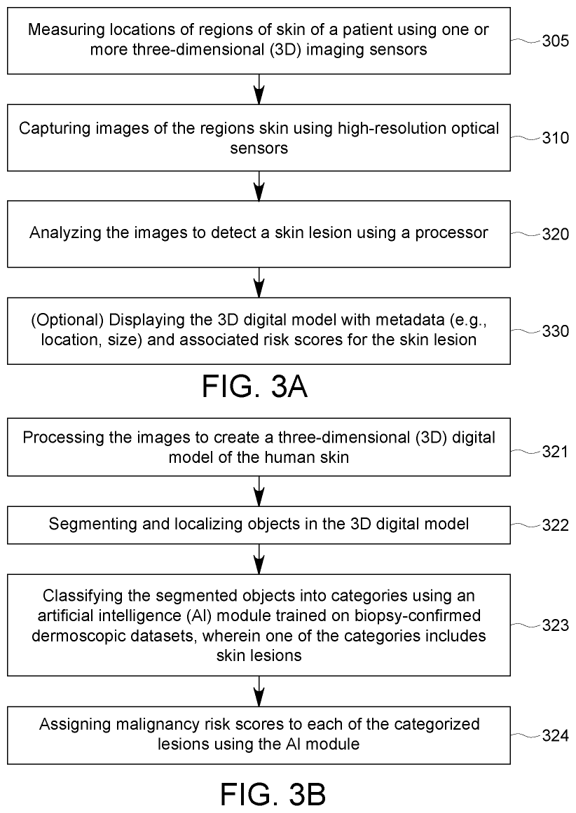

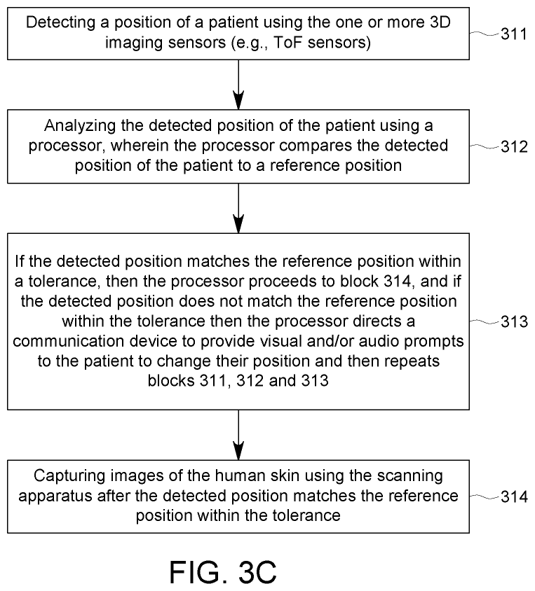

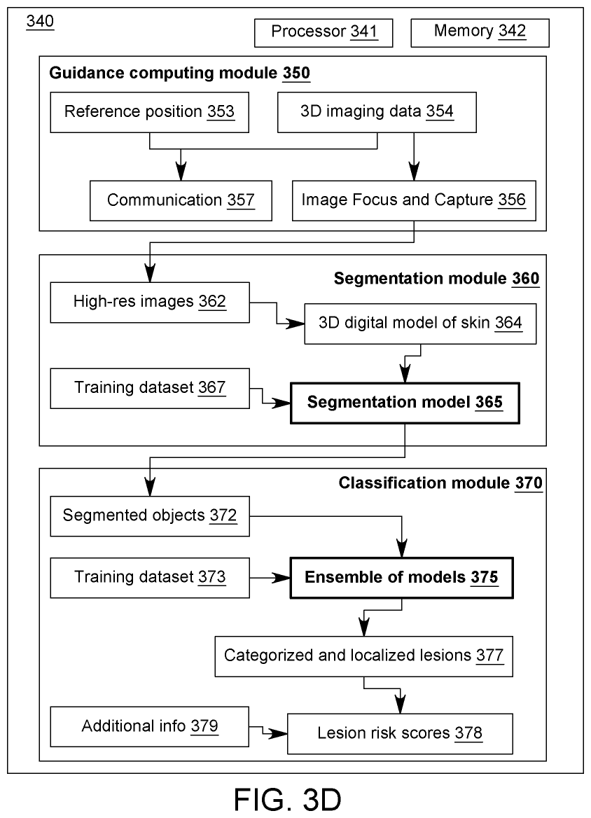

FIGS. 3A-3C show examples of methods for autonomous dermoscopic skin imaging and lesion risk assessment, which can be executed using an autonomous dermoscopic skin imaging and lesion risk assessment system described herein, such as any of the systems shown in FIGS. 1A-2E. The methods in FIGS. 3A-3C can be computer-implemented methods. A processor can read instructions stored in a memory (e.g., random access memory (RAM), read-only memory (ROM), flash memory, solid-state drives (SSDs), or other computer-readable storage media) coupled to the processor, which cause the processor to perform blocks of the methods in FIGS. 3A-3C.

FIG. 3A is a flowchart of an example method for autonomous dermoscopic skin imaging and lesion risk assessment. The method of FIG. 3A may be performed by a local processor, remote processor, or a combination of a local and remote processor. At block 305, locations of regions of skin of a patient are measured using one or more 3D imaging sensors. For example, the 3D imaging sensors can measure the locations of the regions of skin as distances from the 3D imaging sensors to the one or more regions of skin of the patient. In some cases, two or more 3D imaging sensors are used to determine the locations of the regions of skin of the patient. For example, the position of the region of skin can be determined using multi-view stereo between two or more 3D imaging sensors. This information can be transmitted from the 3D imaging sensors to a processor (e.g., processor 106 in FIG. 1A), and the processor can calculate vectors (with lengths and directions) between the regions of the skin of the patient and the high-resolution optical sensors. For example, the processor can use geometrical information about the position of the 3D imaging sensors and the high-resolution optical sensors to convert measured distances between the regions of the skin and each of the 3D imaging sensors into the vectors between the regions of the skin of the patient and the high-resolution optical sensors. The processor can transmit instructions to the high-resolution optical sensors to focus on depths and directions related to the vectors between the regions of the skin of the patient and the high-resolution optical sensors.

At block 310, images of the regions of skin are captured using high-resolution optical sensors (e.g., the high-resolution optical sensors shown in FIGS. 1A-2C). For example, there can be from about 2 to about 100 optical sensors, from about 10 to about 100, or more than 100 high-resolution optical sensors. The processor can be used to instruct the high-resolution optical sensors to capture images of the regions of the skin of the patient in block 310 using focus vectors that correspond to the locations (e.g., distances and directions) determined by the processor using information from the 3D imaging sensors (in block 305). The images may each have a resolution from about 10 microns to about 200 microns, or from about 20 microns to about 25 microns, or less than about 25 microns. The high-resolution optical sensors can be digital cameras including lenses and apertures. In some cases, the optical sensors can include a lens that allows the optical sensor to change the focal length (i.e., the distance at which an object being imaged is in focus). In some cases, the image sensors are from about 10 cm to about 100 cm, or about 30 cm to about 60 cm, away from the skin when capturing the high-resolution images.

For example, in some cases each image sensor is controlled independently in block 310 by the processor. The image sensors can be coupled to a support structure or frame, which can move to capture images from a larger area of the patient (e.g., as shown in FIG. 1C), in some cases. The 3D imaging sensors or depth sensors can be used to create a 3D map of the skin allowing the system to measure the relief of the skin of the patient and create a depth map for each field of view of the image sensors. For example, the processor can use ToF sensor information, structure-from-motion, multi-view stereo, or neural surface reconstruction algorithms to determine the 3D map of the skin based on the information from the 3D imaging sensors. Based on the measured depth maps, the processor can be used to identify one or more areas of skin on which each image sensor can focus. The processor can transmit the coordinates (e.g., in (X, Y) space) or focus vectors (including distance and direction) to focus the field of view of each image sensor. Once the images are taken by the image sensor(s), the processor can be used to discard the areas that are not in focus in one or more images, and reconstruct the image with one or more in-focus patches of one or more other images to generate a composite image of the region of skin that is in focus.

The information from the 3D imaging sensors can be used by the processor to determine which areas can be in focus, and which areas cannot be in focus. For example, the 3D imaging sensors can measure distances to areas of skin. The processor can convert those measured distances into locations in 3D space within the scanning apparatus. Those locations can be compared with known positions and ranges of focal depths for each imaging sensor to determine which areas can be in focus, and which areas cannot be in focus. In some cases, the method in FIG. 3A can further include a block where the processor instructs one or more image sensors to move closer to or farther away from the skin of the patient using a mechanical movement system (e.g., as shown in FIG. 1C), either before or during block 310 (capturing the images of the regions of skin). For example, if the skin of the patient is out of range (too close to, or too far away from, the optical sensor), the image sensor can be moved so that it can focus on the region of skin to obtain a high-resolution image.

At block 320, the processor can analyze the images to detect one or more skin lesions present in the image(s) using a processor. The processor can use one or more AI models to segment, classify, and assign scores to skin lesions or other conditions, as described herein. For example, the processor can use one or more AI models to locate and segment an object in an image of the skin of the patient. The processor can then use another set of AI models to classify each segmented object. An associated risk score can then be assigned to each classified object by the processor, for example, using another set of AI models, an algorithm, or a look-up table. In some cases, the risk score can be derived from the classification of the object and additional information, such as information about the object over time (e.g., from images taken in previous sessions) or other information about the patient (e.g., age, sex, or other relevant history).

At optional block 330, the 3D digital model is displayed with metadata (e.g., location, size) and associated risk scores for one or more objects (e.g., skin lesions), for example, using a monitor display, handheld device, paper printout, email, text, or any type of electronic messaging. In other cases, the results of the imaging and risk assessment are not displayed but rather are communicated electronically to another system that uses the results.

The results of the imaging and risk assessment displayed in block 330 can also be sent to a physician for review. In some cases, the method shown in FIG. 3A can further include a block after block 320, where the processor generates a recommendation for an additional diagnostic or treatment based on the risk score(s) or risk assessment and optionally other parameters. The recommendation can be generated by the processor of the system based on the risk score(s) or risk assessment for the patient, and optionally additional data. In some cases, the method shown in FIG. 3A can further include a block after block 320, where an additional treatment can be performed. A lesion that is detected using the systems and methods described herein (e.g., in block 320 of the method in FIG. 3A) can be treated in several ways. The treatment or recommended treatment can depend on several factors such as the type and size of the lesion, and whether it is benign or malignant, or suspected of being benign or malignant. For example, in the case of benign lesions with no concerning features, then the treatment method block (i.e. after block 320) can include further observation and monitoring, wherein the lesions are tracked for changes over time. In another example, a lesion can have a score that indicates that it is a precancerous actinic keratoses, or an inflammatory lesion, and the treatment method block can include application of topical medicated creams or ointments. In another example, a detected lesion can be treated using cryotherapy, where the lesion is frozen using liquid nitrogen to destroy abnormal cells in the treatment method block. In another example, a detected lesion can be removed using surgical excision, which can be used for lesions that are suspicious or malignant (e.g., melanoma or basal cell carcinoma) in the treatment method block. In another example, a detected lesion can be treated using laser therapy, for example, for vascular or pigmented lesions, or for cosmetic improvement of the skin in the treatment method block. In another example, a detected lesion can be treated using photodynamic therapy in the treatment method block, where light-sensitive drugs and light are used to destroy abnormal skin cells, which can be used for precancerous lesions. In another example, in cases where a detected lesion is malignant, the treatment can also include further oncologic therapies in the treatment method block, such as chemotherapy, radiation therapy, targeted drug therapy, or immunotherapy, depending on the type and stage of cancer. In some cases, the treatment recommendation is provided to a physician who advises the patient on treatment options, for example those described above. For example, in some cases, patients with lesions with high risk scores can be referred to a specialist for further diagnosis or treatment (e.g., those described above), which can be beneficial for the patients to receive timely and accurate diagnoses.

In some cases, the images are analyzed (e.g., at block 320 in FIG. 3A) to detect any skin lesions present by using a processor to perform the method operations shown in FIG. 3B. At block 321, the images are processed using information from the 3D imaging sensors (in block 305) to create a 3D digital model of the human skin. For example, focus stacking can be used to combine images taken using different focus depths into a 3D rendered model of the skin (as described further herein). At block 322, objects in the 3D digital model are segmented and localized. The segmentation and localization can be done using segmentation AI models, such as foundation computer-vision models and/or task-specific segmentation models such as YOLOv11, SAM2, and/or DINOv2. The segmentation AI models used in block 322 can be trained computer vision models that operate on image pixels to learn patterns of color, texture, and/or shape that correspond to skin lesions. In some cases, the segmentation AI models can analyze segmented images containing lesions or other skin conditions and output a probability mask that delineates a lesion or skin condition. The segmentation AI model(s) (e.g., a convolutional or transformer-based segmentation network) can analyze local neighborhoods of pixels across multiple scales. They can learn to recognize signals, such as intensity and/or color distributions, texture, edges, and shape/irregular borders. The output is a per-pixel probability map, and thresholding the probability map can yield the lesion boundary.

The lesion boundary (e.g., determined in block 322) can be used to determine the location of the lesion, and other attributes (e.g., size and shape) of the lesion at one point in time or to track changes over time. For example, a first 3D model of the skin of a patient can be created at a first instance in time, and a first lesion boundary of a lesion can determined at the first instance in time. A second 3D model of the skin of the patient can be created at a second instance in time after the first instance in time (e.g., 1 week, 1 month, 6 months, or 1 year after), and a second lesion boundary of the lesion can determined at the second instance in time. The processor can be used to determine a difference between the first and second lesion boundaries and use the difference to determine a risk score for the lesion. For example, the difference can include a difference or change in the area, size, shape, or the degree of border irregularity (e.g., the degree to which the boundary is categorized as jagged, uneven, notched, scalloped, or blurred).

The AI models used to segment the images in block 322 can be trained on a large dataset of annotated skin lesions and skin conditions. For example, the training dataset can be manually labeled, or can include some or all synthetic images (as described further herein). At block 323, the segmented objects are classified into categories using a classification AI module (e.g., using an ensemble of image-classification models, such as those including EfficientNet-V2 backbones (with different width/depth/resolution settings), or 3 EVAv2-based vision transformers). The AI classification model(s) can be trained on biopsy-confirmed dermoscopic datasets. For example, the training dataset can be manually labeled, or can include some or all synthetic images. One of the classification categories can include skin lesions, and in some cases, the classification categories can also include subcategories of different types of skin lesions (e.g., melanocytic nevus, melanoma, basal cell carcinoma (BCC), and squamous cell carcinoma (SCC), hemangioma). Other classification categories can include other types of skin features that are not lesions, such as freckles, moles (nevus), skin tags, and seborrheic keratoses. Other types of skin conditions can also have classification categories so that they can be detected using the systems and methods described herein, for example, acne, rosacea, vitiligo, eczema, psoriasis, etc.

At block 324, the classification AI module assigns a malignancy risk score to one or more categorized lesions. The classification AI module can incorporate AI models trained to learn visual patterns characteristic of a lesion class as well as to compute calibrated class probabilities from input images. These probabilities can be mapped to a risk score for each detected and categorized lesion. In some cases, the classification AI module (block 323) can categorize detected objects into clinically relevant classes such as melanocytic nevus, melanoma, basal cell carcinoma (BCC), squamous cell carcinoma (SCC), hemangioma, seborrheic keratosis, and others. The predicted category may determine the risk score in some configurations. In other cases, additional information may be incorporated, for example, the rate of growth of a lesion over time, which can be derived from segmented and categorized lesions observed at different visits. For example, an object can be classified as a non-cancerous object (e.g., a freckle or a benign mole), and be given a very low or zero risk score, while an object that is classified as a cancerous lesion would be given a high risk score (e.g., greater than 0.8 out of a possible 1.0), depending on classified attributes of the lesion (e.g., classification category, size, etc.).

In some cases, a model can output multiple scores for a single lesion. For example, a class-membership probability score can be output for a given lesion and one or more abnormality or atypicality scores can also be output for the same lesion. For example, a class-membership probability score can be a model-estimated probability that a lesion belongs to a given diagnostic class. In the case of a single-label setup, the per-class probability score values would sum to 1. An atypicality or abnormality score is the degree of how unusual the lesion looks (e.g., compared to other objects detected on the skin of the same patient), independent of any specific class malignancy score. The overall malignancy scores can estimate the probability of malignancy, either from a dedicated malignancy score, or by summing class probabilities over malignant and premalignant classes (e.g., a melanoma 0.30+BCC 0.02 yields an aggregate score of 0.32). In some cases, boosting (e.g., using a meta-model) can be used to determine some of the scores, and not other types of scores. For example, boosting can be done for the models used to determine the class-membership estimate, but not for the models used to determine the abnormality scores.

FIG. 3C shows an example of a method for autonomous positioning of a patient during an autonomous dermoscopic skin imaging and lesion risk assessment (e.g., as shown in the methods in FIGS. 3A and 3B). At block 311, a position of a patient is detected using 3D imaging sensors (e.g., ToF sensors, or stereoscopic sensors). For example, a proximity sensor can be used to detect the presence of a person in the system and trigger the 3D imaging sensors to acquire data of the 3D position of the patient or regions of skin of the patient. In another example, a patient can use a voice command or press an input element (e.g., button, slider, toggle, etc.) associated with the system to trigger the 3D imaging sensors in block 311. In some cases, the trigger can initiate a countdown before the 3D sensing starts. At block 312, the detected position of the patient is analyzed using a processor, where the processor compares the detected position of the patient to a reference position (e.g., an ideal position, or a position of the same patient measured in a previous session). In block 313, if the detected position matches the reference position within a tolerance, the processor proceeds to the operation in block 314. For example, the tolerance can be a fixed length (e.g., 10 cm), or a percentage error (e.g., within 10%) of the distance between the image sensor and the skin. However, if the detected position does not match the reference position within the tolerance, the processor may direct a communication device (e.g., audio speakers and/or a visual display) to provide visual and/or audio prompts to the patient to change their position (e.g., change their posture, or positioning, or position of a particular body part). After the patient has changed their position according to the prompts, the method repeats operations in blocks 311, 312, and 313. In block 314, after the detected position of the patient (in block 311) matches the reference position within the tolerance, the images of the human skin are captured using the scanning apparatus (e.g., in block 310 of the method in FIG. 3A). In some cases, there are a maximum number of iterations (e.g., from 2, 3, 5, or from 3 to 10) where the method can complete block 313 and return to block 311. After the maximum number of iterations is reached, then the system can communicate a message to the patient, such as to exit the scanning apparatus and start again using a different position of their body, or to start again using a different configuration of the scanning apparatus (e.g., to utilize a step stool or other mechanical body support provided by the system, or select another mode of operation of the system).

In some cases, the system includes an image quality pipeline that verifies the quality of the images captured by the image sensors in real time. In cases where the image quality pipeline determines that the image is not of good quality (e.g., has a resolution less than about 200 microns, or less than about 25 microns, or less than about 20 microns), the processor can instruct the system to acquire another image. In some cases, the processor can instruct one or more image sensors to move (e.g., move closer to or farther away from the skin) before capturing another image. In some cases, the processor can instruct the guidance system to instruct the patient to move before capturing another image. In some cases, the image sensors contain an autofocus.

FIG. 3D shows an example of a computing system 340, including a processor 341 and computer memory 342. The processor 341 can be communicatively coupled to the computer memory 342, for example, through a wired connection (e.g., a data bus) or wirelessly through one or more protocols. The computing system 340 can be part of the systems shown in FIGS. 1A-2E. The processor 341 can be the same processor as processor 106 in FIGS. 1A-1C, or it can be a different processor (e.g., in the cloud or remote). The computing system 340 can be used to perform the methods described herein, for example, the methods shown in FIGS. 3A-3C. The computing system 340 includes a guidance computing module 350, a segmentation module 360, and a classification module 370.

For example, processor 341 can include one or more central processing units (CPUs), graphics processing units (GPUs), tensor processing units (TPUs), digital signal processors (DSPs), neural processing units (NPUs), field-programmable gate arrays (FPGAs), application-specific integrated circuits (ASICs), or other computer processors. For example, computer memory 342 can include random access memory (RAM), read-only memory (ROM), cache memory, flash memory, non-volatile memory, solid-state drives (SSDs), magnetic storage devices, optical storage media, or other computer-readable storage media.

The guidance computing module 350 includes 3D imaging data 354 obtained using the 3D imaging sensors (e.g., 3D imaging sensors 112 in FIGS. 1A and 1B). The 3D imaging data 354 can be provided to an image focus and capture module 356, which can determine instructions (e.g., coordinates or focus vectors) for high-resolution optical sensors to focus on particular depths and directions to capture images. The instructions can then be provided to the high-resolution optical sensors to capture the high-resolution optical images 362 (e.g., with resolution from about 10 microns to about 200 microns, or from about 20 microns to about 25 microns, or less than about 25 microns). The high-resolution images 362 captured using the high-resolution optical sensors (controlled by or using input from the image focus and capture module 356 of the guidance computing module 350) can be transmitted to the segmentation module 360.

The guidance computing module 350 also includes an input reference position 353 of the patient, for example, measured in a previous session (as described herein). The reference position 353 can be compared to 3D imaging data 354 obtained using 3D imaging sensors (e.g., 3D image sensors 112 in FIG. 1B). When the reference position 353 matches a position of the patient determined using the 3D imaging data 354 within a tolerance (as described herein), the guidance module can trigger the image sensors to acquire the high-resolution images 362, to be provided to the segmentation module 360. When the measured 3D imaging data does not match the reference position 353 within the tolerance, an instruction is provided to the communication module 357 to communicate one or more prompts to the patient, as described herein. In some cases, the system can include a proximity sensor that can measure a patient in the measurement area and transmit a signal to the guidance computing module 350 to start collecting 3D imaging data 354.

Segmentation module 360 processes the high-resolution images 362 and creates a 3D digital model 364 of the skin using one or more AI models, as described herein. A segmentation AI model 365 can be trained on a training dataset 367 and used to extract segmented objects 372 (e.g., lesions, moles, skin tags, etc.) from the 3D digital model 364 of the skin. The AI models that can be used to produce the 3D digital model 364 and those in the segmentation AI model 365 can be any of those described herein. For example, the segmentation AI model 365 can include foundation computer-vision models and/or task-specific segmentation models such as YOLOv11, SAM2, and/or DINOv2.

The classification AI module 370 processes the segmented objects 372 using an ensemble of AI models 375, which can be trained on training dataset 373. The ensemble of AI models 375 can classify the segmented objects 372 and produce a set of categorized and localized lesions 377. The category of the lesion can be used to determine a lesion risk score 378. In some cases, additional information 379 can be used in conjunction with the set of categorized and localized lesions to determine the lesion risk scores 378. For example, an ensemble of AI models 375 can include image-classification models with EfficientNet-V2 backbones (with different width/depth/resolution settings), or EVAv2-based transformers, trained with slightly different combinations of widths, depths, resolutions, hyperparameters, and/or random seeds.

Computing system 340 can perform the methods described herein. For example, localization of the segmented objects 372 or features can be performed by the segmentation model 365. The lesion can be identified on the 3D model 364 or body map from the segmentation output 372, and the categorized and localized lesions 377 (e.g., with localized position and other information) can be saved in the meta-data for the image of the segmented object. The ensemble of AI models 375 can consume the localized and segmented lesion 377 and assign a diagnostic category or risk score 378.

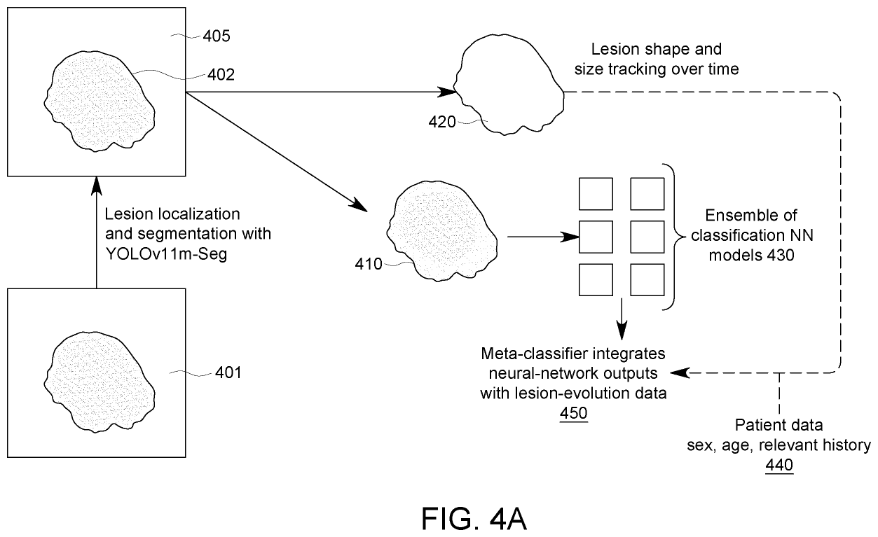

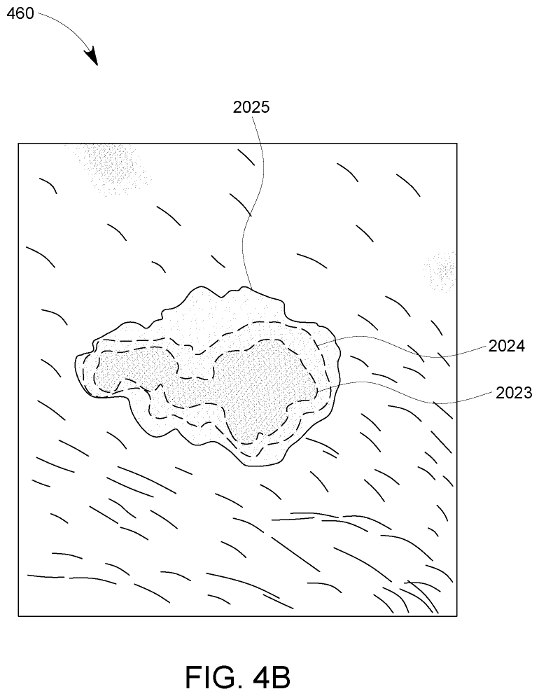

FIG. 4A shows an example of a method for dermoscopic skin imaging and lesion risk assessment. The method in FIG. 4A can be executed using autonomous dermoscopic skin imaging and lesion risk assessment system, such as any of the systems shown in FIGS. 1A-2E. In this example, a high-resolution image 401 is segmented and lesions are localized using a first AI model. In this example, a boundary 402 of a lesion is determined using a segmentation module (e.g., segmentation module 360 in FIG. 3D) described herein and overlaid on the high-resolution image of the lesion in image 405. Another AI model is used to classify the segmented image 410. The AI model can include an ensemble of classification neural network (NN) models 430 based on EfficientNet-V2, EVAv2, and/or other state-of-the-art image-classification architectures. In some cases, ensembles can be used to improve the accuracy of the AI model by reducing the variance component of prediction error. Additionally, in this example, the size and/or the shape 420 of the segmented lesion image is extracted from the segmented image. In this example, the extracted size and/or shape of a detected lesion is tracked over time (e.g., by performing the method shown in FIG. 4A periodically over time). A meta-classifier incorporates information including the AI model classification of the lesion from the segmented image 410, the size and/or shape 420 of the lesion over time, and optionally 440 patient data (e.g., sex, age, relevant history). The output 450 from the meta-classifier can include location, size, and/or a risk assessment score (e.g., which can be expressed either as a probability that a lesion belongs to a specific diagnostic class or a measure of how atypical the lesion is relative to the patient's other lesions) for any lesions detected. The output 450 can optionally be displayed as described herein. The output 450 can also optionally be used to determine a further treatment as described herein.

FIG. 4B shows an example of a lesion 460 that is tracked over time. This example shows a high-resolution image of the lesion 460 in 2025 that shows a boundary or an outline indicating lesion shape and/or size over time (labeled as (in “2023,” “2024,” and “2025” in the figure). In this case, the lesion shape and/or size was tracked in three separate measurements taken over three consecutive years (in 2023, 2024, and 2025), or over the span of about two years. In this example, lesion 460 increased in area over the range of about two years. The aspect ratio of the boundary of lesion 460 also changed from higher length-to-width ratio to lower length-to-width ratio, for the “2023” boundary compared to the “2025” boundary.

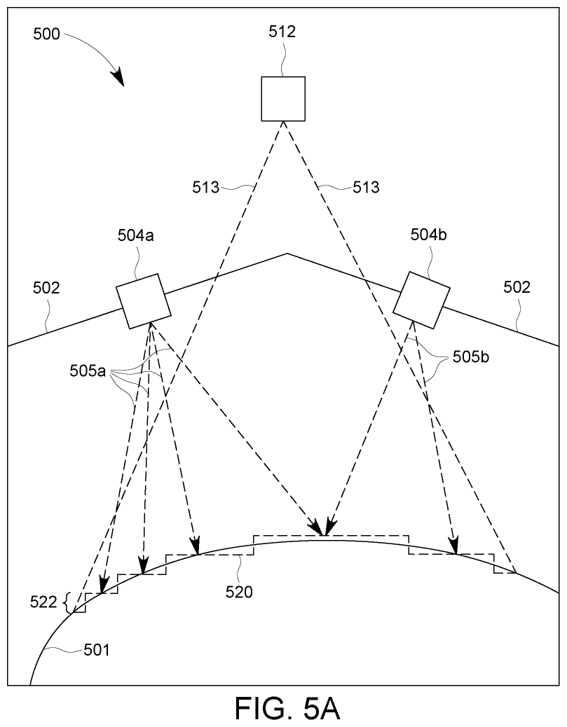

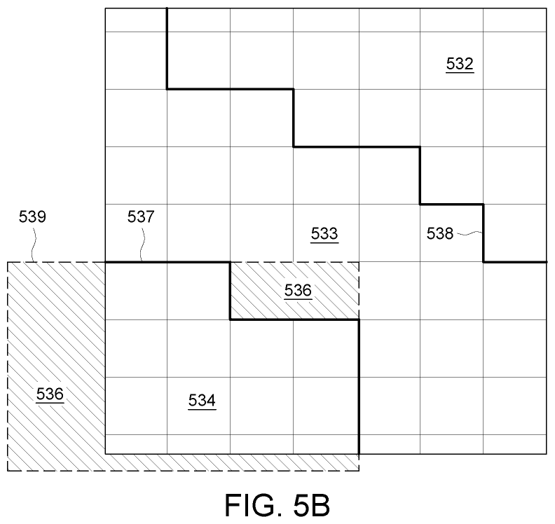

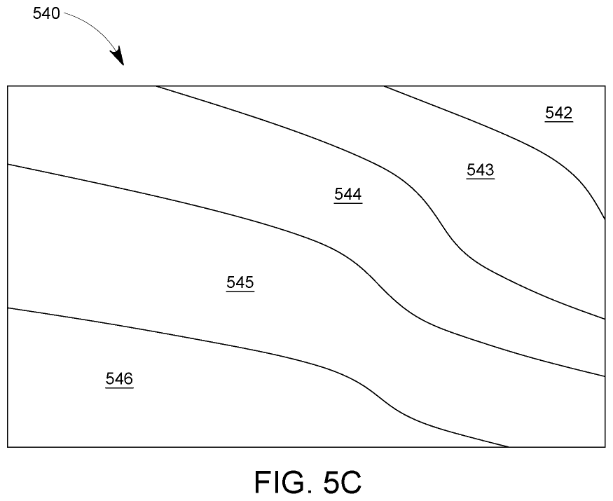

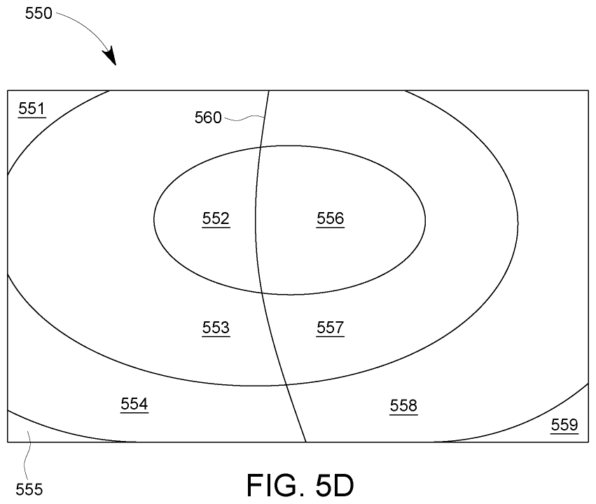

FIG. 5A shows a schematic of an example of a portion of a system 500 that can be used to acquire images of a 3D surface 501 using image sensors 504a, 504b coupled to a support structure 502 (e.g., as shown and described with respect to image sensors 112 and support structure 122 in FIGS. 1A and 1B). The image sensors 504a, 504b each have a field of view (FOV) and a depth-of-field (DOF), and they can be focused at particular points in space, for example, as shown by the focus vectors 505a, 505b, respectively. 3D imaging sensor 512 can be used to measure the position of the region of skin within a certain FOV 513. In some cases, the 3D imaging sensor 512 can map a grid or mesh of positions 520 to approximately represent the 3D surface 501 of the skin of the patient. For example, the grid or mesh of positions 520 can have step heights 522 that are smaller than a DOF of the image sensors 504a, 504b. High-resolution image sensors have a DOF (e.g., a few millimeters), which means that they can only capture a certain region of a curved surface at high resolution (i.e., without going out of focus).

The image sensors can therefore be focused on different depths and directions (according to vectors 505a, 505b in this example) to capture high-resolution images of curved surface 501. In some cases, multiple image sensors 504a, 504b can be used to capture a large portion of the 3D surface 501 while staying in focus to capture high-resolution images across a large spatial area (e.g., greater than about 50 cm², greater than about 100 cm², greater than about 70% of the skin of the patient, greater than about 80% of the skin of the patient, or greater than about 90% of the skin of the patient). The processor can be used to form a composite image from sub-images taken using the image sensors 504a, 504b. Each sub-image can be a portion of an image taken, keeping regions of the image that are in focus. In other words, within each sub-image making up the composite image, the distances or magnitudes of vectors 505a, 505b between the image sensors 504a, 504b and the curved surface 501 are relatively constant and within a DOF of the image sensor. This enables high resolution across each sub-image, and by combining multiple sub-images together, across a large spatial area of the composite image.

FIGS. 5B-5D show examples of top-down views of 3D digital models that can be made using any of the systems and methods described herein, for example, any of the systems in FIGS. 1A-2E and 5A.

In FIG. 5B, there are three regions 532, 533, 534 that correspond to different positions or regions of the skin of the patient, or correspond to different focus vectors (e.g., 505a, 505b in FIG. 5A) between the skin of the patient and the high-resolution optical sensors (e.g., 504a, 504b in FIG. 5A). For example, region of skin 532 can be farthest away from a high-resolution optical sensor, region of skin 533 can be closer to the high-resolution optical sensor, and region of skin 534 can be closest to the high-resolution optical sensor. The example in FIG. 5B includes a grid or discretized mesh of focus distances, with boundaries 537, 538 between the regions of skin 532, 533, 534, each of which has a focus distance associated with it.

For example, a processor of a system or method described herein can determine focus vectors that correspond to regions 532, 533, 534 of the skin of a patient, for example, using 3D imaging or depth sensors. Note that region 533 overlaps with region 536. The processor can then be used to instruct one or more image sensors to capture images using focus vectors pointing at each of the regions. The processor can then crop out areas of each sub-image that are out of focus, based on the information from the 3D imaging or depth sensors. For example, an image sensor can capture an image shown by the rectangular dotted region 539, using a focus vector pointing to a location within region 534. The processor can then crop out area 536 (which is a discontinuous area, in this example) that is out of focus, based on the information from the 3D imaging or depth sensors. The processor can form the composite image including regions 532, 533, 534 from a set of sub-images that are each in focus (e.g., using focus stacking). The areas of each captured sub-image that are out of focus (e.g., area 536 of image 539) can be discarded, and the regions that are in focus (e.g., region 534 of image 539) can be used in the composite image.

FIGS. 5C-5D each show multiple regions of skin 542-546, and 551-559, respectively, each with an approximately constant focus distance across the area of the region, such that each region can be in focus and used to form the composite images 540, 550 shown in the figures. For example, each of regions 542-546 and 551-559 can have a focus distance that is within a DOF (e.g., DOF 522 in FIG. 5A) throughout the region, such that the image sensors can capture images of these regions at high resolution. FIGS. 5B and 5C show examples where the boundaries between the regions of skin 532-534 and 542-546 are approximately diagonal across the region of the 3D model shown, as could be the case if the region of skin were sloped in one direction or undulating.

FIG. 5D shows an example where the boundaries between the regions of skin 551-559 are approximately conic sections (e.g., ellipses, or parabolas) across the region of the 3D model shown. For example, FIG. 5D could illustrate a protrusion of a region of skin in 3D such as on a shoulder, breast, or elbow of a patient. The boundary line 560 can represent a boundary between images taken by a first high-resolution optical sensor (e.g., 504a in FIG. 5A) and a second high-resolution optical sensor (e.g., 504b in FIG. 5A). In other words, regions 551-555 can be captured using the first high-resolution optical sensor (e.g., 504a in FIG. 5A) and regions 556-559 can be captured using the second high-resolution optical sensor (e.g., 504b in FIG. 5A). These examples show that a 3D model of the skin of a patient can be created using multiple high-resolution images taken using different focus vectors, and in some cases, using multiple high-resolution optical sensors each focusing on two or more different depths.

In some cases, one or more of the high-resolution optical sensors are stationary; however, they have adjustable focal lengths, which allow them to capture multiple images, which can be combined using a processor into a composite image (e.g., using focus stacking), thereby allowing them to capture a larger portion of the 3D surface 501 while staying in focus to provide high resolution across a large spatial area. By changing the focal distance between the image sensor(s) and the curved surface, high-resolution images can be captured across a large spatial area without moving the image sensor.

In some cases, the focal length of one or more of image sensors can be changed, and one or more of the image sensors can be moved to capture a large area of a patient's skin at high resolution (e.g., as shown in FIG. 1C). For example, a high-resolution optical sensor can be moved if the distance between the skin of the patient and the sensor is below a predefined threshold or threshold range or above a predefined threshold or threshold range, such that the sensor is unable to focus. Additionally, if changing the focal length, and optionally changing position of the image sensor, is not sufficient due to the patient's body positioning, the guidance module can instruct the patient to move their body into a measurable position.

The multiple images shown in FIGS. 5B-5D can be combined into the 3D models or maps shown in the figures using focus stacking techniques, which can be used to combine the images and determine a 3D model including the measured approximation of the 3D surface 501. The 3D model including the multiple high-resolution images can be analyzed by the classification module (e.g., in method block 323 of FIG. 3B), and the information in the 3D model can be used by the segmentation and classification AI modules to segment, localize, and classify an object in the composite image.

In some cases, only the portion or portions of the images taken using a high-resolution optical sensor (e.g., 504a, 504b in FIG. 5A) that are in focus are saved, and the rest of the image (e.g., area 536 in FIG. 5B) is not saved or analyzed. This can beneficially save computing resources. For example, a high-resolution optical sensor can be instructed to capture an image (e.g., over area 539 in FIG. 5B) with a focus vector corresponding to region 534 in FIG. 5B. In some cases, the pixels in that image that correspond to region 534 are saved and analyzed by the system, and the pixels in area 536 that are outside of region 534 in that image are not saved, since they are assumed to be out of focus. This process can be performed multiple times such that all areas of a region can be captured and saved to be combined in the 3D model. In some cases, the regions that are saved and analyzed in an image are not contiguous. For example, regions 551 and 555 in FIG. 5D could have the same focus depth and be captured by a high-resolution optical sensor in an image, so that those regions are saved to be used to form the 3D model shown in FIG. 5D. The regions outside of regions 551 and 555 that are discarded can also be non-contiguous (e.g., area 536 in FIG. 5B). In such cases, a focus vector corresponding to region 551 or a focus vector corresponding to region 555 can be used to capture the image from which regions 551 and 555 pixels are saved and analyzed.

Distance sensors, 3D image sensors, or depth sensors, can be used to determine the distance (e.g., distance 140 in FIG. 1C) from the image sensor to the skin and provide active feedback to the processor. The processor can use the information from a distance sensor to instruct an image sensor movement system (e.g., image sensor movement system 132 in FIG. 1C) to move the image sensor to a predefined distance (or to within a predefined distance range) from the skin. In some cases, the systems and methods described herein can include multiple image sensors, each coupled to a movement system, and each having a distance sensor, 3D image sensor, or depth sensor. In such cases, a processor can instruct one or more of the movement systems where to move the image sensors so that the image sensors are within a predefined distance of a patient's skin. For example, such movement can be towards or away from the skin, or it can be lateral or rotational movement to move the image sensor such that the distance between the image sensor and the skin is within the depth of field of the sensor, and it is pointed at the region of skin to be imaged. In some cases, the movement systems described above may not be able to move the image sensors enough, due to the position of a patient's body. In such cases, the guidance module can instruct the patient to move their body into a region within a depth of field (DOF) of one or more image sensors so that region of skin can be measured at high resolution. In some cases, before instructing the image sensors to move, the guidance module can instruct the patient to move their body into a region within a DOF of one or more image sensors, based on information from the distance sensors.

In some cases, the systems and methods described herein can acquire images of greater than about 50%, greater than about 75%, or greater than about 90% of the area of a patient's skin at high resolution (e.g., from about 10 microns to about 200 microns, or from about 20 microns to about 25 microns, or less than about 25 microns), for example, using multiple cameras, each one equipped with movement capabilities.

### AI Segmentation and Classification

In the segmentation block (e.g., block 322 in FIG. 3B), a segmentation AI model is used to segment skin lesion(s) in an image, produce a segmentation mask, and crop out the image of the localized lesion from the image background before passing it to the classifier. For example, a modified YOLOv11m-Seg, a SAM2, or DINOv2 model can be used to segment the images. The segmentation AI model can be trained using a dataset of images including images of skin, skin lesions, and other skin conditions.

In some cases, the segmentation AI model is trained on a mixed corpus. The core of the dataset can include an initial dataset of macro photographs (e.g., with magnifications of about from about 1× to about 10×) of skin (e.g., from a publicly available database) that are annotated. In some cases, the annotation can be done by hand, for example, if the original dataset contains errors or lacks segmentation masks. These initial images can be supplemented with additional images (macro shots plus medium resolution dermoscopic images). These additional images can be annotated. In some cases, the additional images can be assigned higher sampling weights than the initial images during training, for example, if they better reflect the conditions in which the device may operate. For example, such a training method can be beneficial if the initial datasets are relatively low resolution, while the images used in the systems and methods described herein are higher resolution.

Additionally, images of rare lesion types that are under-represented in real macro data (e.g., basal-cell carcinoma) can be added to the training dataset by generating synthetic images (e.g., using Stable Diffusion). In some cases, the synthetic images may have artifacts, and those images can be discarded. The synthetic images can be annotated and used in the dataset to enlarge the rare-class sample, which can be beneficial to improve the accuracy of detection of objects in the rare class.

In some cases, the systems and methods described herein, including the segmentation model training described above, can improve generalization to weakly pigmented and non-pigmented lesions.

In the classification block (e.g., block 323 in FIG. 3B), the segmented objects are classified into categories using a classification AI module that is trained on biopsy-confirmed dermoscopic datasets. The input to the classifier is the cropped, localized lesion image produced by the segmentation block described above. The classification AI module can include one or more neural network-based models, an ensemble of image-classification models, EfficientNet-V2 backbones (with different width/depth/resolution settings), or EVAv2-based vision transformers. For example, the classifier can be an ensemble of six image-classification models including EfficientNet-V2 backbones (different width/depth/resolution settings), and EVAv2-based vision transformers.

The training strategy for the segmentation AI module and/or the classification AI module (e.g., segmentation module 360 and/or the classification module 370 in FIG. 3D) using respective training datasets 367 and 373 can include one or more of:
Data splits. In some cases, each model is trained on a distinct, partially overlapping fold created with stratified group k-fold to keep all images from a single patient in the same fold and preserve lesion-type proportions. “Stratified group k-fold” is a method wherein each fold in k-fold cross-validation maintains the same class distribution as the overall dataset and also considers group structures within the data. Additionally, “out of fold” refers to the data that is not included in a specific fold during training.
Class imbalance. In some cases, mini-batches are balanced on-the-fly, ad hoc, as needed, or in real time; minority classes can be up-sampled, and majority classes can be down-sampled.
Synthetic augmentation. In some cases, a similar synthetic-image generation pipeline used in segmentation is used to bolster under-represented classes (e.g., actinic keratosis or basal-cell lesions) in training data.
Weight smoothing. In some cases, during training, an exponential moving average (EMA) of the weights is maintained. In such cases, the EMA checkpoints can be the ones used in inference due to their better performance

In some cases, the lesion class can be determined by a weighted vote across the ensemble. For example, weights can be based on model performance on out-of-fold (OOF) data. Alternatively, the final class can be produced by a gradient-boosted meta-model (GBDT) (e.g., XGBoost, LightGBM, CatBoost) that ingests per-model predictions together with auxiliary features.. The GBDT meta-model can include one or more of:
probability scores from each ensemble network;
visual features (e.g., handcrafted visual features) derived from the segmentation mask (shape descriptors, color variance, border irregularity);
patient metadata (e.g., sex, age, Fitzpatrick skin type, relevant history, etc.); and
lesion evolution, including changes in size and shape observed over time (e.g., across follow-up scans).

Incorporating lesion evolution temporal features can also be injected into the meta-classifier, which can be beneficial to further improve diagnostic accuracy.

In some cases, synthetic data can be generated and used in training datasets used to train the segmentation and classification AI models described herein. In some cases, a custom pipeline for generating synthetic skin-lesion images (e.g., using Stable Diffusion 1.5) can be used. The synthetic data generation can include one or more of the following:
Generative backbone. Synthetic images can be generated with fine-tuned generative AI tools—for example, text-to-image latent-diffusion models such as Stable Diffusion 1.5, Stable Diffusion XL, or Flux—but are not limited to latent-diffusion approaches (e.g., diffusion transformers, autoregressive, or GAN-based models).
Prompt-driven sampling. Using the methods described above, the fine-tuned model can be used to produce large batches of synthetic images from class-specific prompt templates. The prompt parameters can vary features of the synthetic images such as lesion type and count, hair density, pigmentation, skin tone, and lighting. These prompts can be used to generate a dataset of skin and lesions providing broad coverage of clinically relevant skin and lesion variation.
Half-automated triage. Each synthetic image can be analyzed by the segmentation network (as in the segmentation block described above). In some cases, images whose lesions are not cleanly delineated are flagged for manual review or can be automatically discarded. The survivors, i.e., those images that have not been discarded in a previous operation, are checked by scoring them using the classification AI module (e.g., the classification ensemble). If a predicted class of an object in a synthetic image disagrees with the intended class beyond a probability threshold, the image is rejected and not used in the training dataset. This can beneficially remove mismatches to prevent the synthetic distribution from drifting away from real data and improves the quality of the synthetic images in the dataset.
Fold-aware integration. In some cases, a separate generative model is trained for every cross-validation fold, using only that fold's training split. In such cases, synthetic images will not expose validation data and do not induce leakage, which can beneficially reduce the risk of overfitting.

The curated synthetic photos can be mixed with real images in the training datasets used to train the AI models in both the segmentation and classification blocks. This can beneficially improve class balance—especially for under-represented lesions such as basal-cell carcinoma and amelanotic melanoma—and can boost recall or the accuracy of a model to classify objects (especially in rare categories) without degrading overall performance.
Sub-Dermal Imaging. In some cases, the systems and methods described herein can use image sensors that are sensitive to wavelengths that allow them to image features beneath the surface of the skin. For example, the image sensors can use IR light or UV light to capture high-resolution images of sub-dermal features. These sub-dermal images can be segmented, classified, and displayed in the same way as any image of the skin described herein.
Hybrid AI-Clinician Workflow. In some cases, an initial triage is performed using the automated AI-based lesion identification and risk assessment systems and methods described herein. This initial processing can be reviewed by a physician confirming or updating the data and generating a report for a patient. High-risk lesions identified using the systems and methods described herein can also (e.g., subsequently) be referred for specialist review in person, which can be beneficial for the patients to receive timely and accurate diagnoses.
Updates. The systems described herein can be updated, for example, through software updates or cloud-based AI improvements to maintain their accuracy and effectiveness. For example, the AI models can be trained to include new categories of lesions, or the model detection accuracy can be improved using updated training datasets.

The following are technical features of an example of an autonomous skin health imaging and analysis system and method, including a scanning apparatus coupled to a processor which can control the scanning apparatus and analyze images taken by the scanning apparatus.
1. The scanning apparatus can include a configurable array of high-resolution optical sensors capable of dermoscopic imaging with a resolution between about 20 microns and about 25 microns. The sensors may be integrated into a vertical ring that moves up and down, or into a horizontal gantry above a patient platform. Multiple views may be captured, for example, to achieve complete coverage of the human body.
2. The imaging system can be equipped with polarized lighting and standardized illumination control to minimize glare and optimize contrast of pigmented and non-pigmented lesions. The scanner may operate in various lighting conditions, including ambient environments found in MedSpas or dedicated clinics.
3. Patients can be guided through the scanning process by a user-facing interface that provides voice and visual prompts to ensure proper posture and alignment for accurate imaging, reducing variability between sessions and supporting longitudinal analysis.
4. Once images are captured, a processor can reconstruct a three-dimensional model of the patient's skin surface using photogrammetric techniques such as structure-from-motion and multi-view stereo. The processor can stitch overlapping images and registers anatomical landmarks for consistent scan comparison over time.
5. Lesions can be detected and segmented using computer vision algorithms that extract features such as color variation, asymmetry, border irregularity, and size. An artificial intelligence engine trained on biopsy-confirmed dermoscopic datasets can assign a malignancy risk score to each lesion.
6. A clinical web portal can enable asynchronous review of risk assessments by licensed dermatologists or clinical staff. The interface may support lesion annotation, patient communication, referral management, and/or comparison with previous scans.
7. Patients may interact with the system using a mobile or web application that allows them to book scans, view results, receive alerts, and share reports with physicians, thereby enabling secure storage and continuity of personal skin health data.
8. The scanner can be deployed in a variety of settings, including dermatology clinics, MedSpas, primary care offices, imaging centers, and pharmacies. The scanner may be installed in multipurpose exam rooms or dedicated scanning cabins designed for privacy and optimized imaging.
9. Alternate embodiments include a handheld dermoscopic scanner for point-of-care use and a semi-mobile unit for modular deployment in compact environments.
10. The disclosure supports a hybrid AI and clinician workflow in which initial triage is automated, but high-risk lesions can be referred for specialist review. The system may be designed to scale through software updates and cloud-based AI improvements.

The following are examples of autonomous skin health imaging and analysis systems and methods, including a scanning apparatus coupled to a processor that can control the scanning apparatus and analyze images taken by the scanning apparatus.
1. An example system for autonomous dermoscopic skin imaging and lesion risk assessment including a scanning apparatus including a plurality of high-resolution optical sensors configured to capture overlapping images of the human skin surface at a resolution between twenty and twenty-five microns, a guidance module configured to direct patient posture and positioning during image acquisition via visual and/or audio prompts, a processing engine configured to construct a three-dimensional digital skin model by applying photogrammetric reconstruction techniques to the overlapping images, a lesion detection module configured to identify, segment, and localize skin lesions on said three-dimensional model, an artificial intelligence module trained on biopsy-confirmed dermoscopic datasets to assign malignancy risk scores to each identified lesion, and a clinician interface configured to display the digital skin model with lesion metadata and risk scores for clinical review and decision support.
2. The system of example 1, wherein the scanning apparatus is operable by a patient without technician assistance using autonomous calibration and motion control.
3. The system of any one of the preceding examples, but particularly example 1, wherein the scanning apparatus includes a motorized vertical ring or gantry that moves around the patient during scanning.
4. The system of any one of the preceding examples, but particularly example 1, wherein the three-dimensional digital skin model is generated using structure-from-motion, multi-view stereo, or neural surface reconstruction algorithms.
5. The system of any one of the preceding examples, but particularly example 1, wherein the three-dimensional digital skin model is used to track lesion progression over time including comparisons of size, shape, color, and AI risk score across multiple scans.
6. The system of any one of the preceding examples, but particularly example 1, wherein the clinician interface supports asynchronous review of triaged lesions, structured diagnostic feedback, and integration with referral workflows.
7. The system of any one of the preceding examples, but particularly example 1, wherein the lesion detection module includes clustering of high-risk lesions based on anatomical zones, sun exposure patterns, or lesion density.
8. The system of any one of the preceding examples, but particularly example 1, further including a patient-facing interface configured to allow scan booking, access to historical reports, and secure messaging with providers.
9. The system of any one of the preceding examples, but particularly example 1, wherein the processing engine applies patient-specific templates to guide consistent posture and lighting across sequential scans.
10. The system of any one of the preceding examples, but particularly example 1, wherein the scanning apparatus is configurable for use in either a vertical orientation allowing the patient to stand or rotate during image acquisition or a horizontal orientation allowing the patient to lie down during scanning.
11. The system of any one of the preceding examples, but particularly example 1, wherein the scanning apparatus is installed either within a multipurpose examination room or within a dedicated scanning cabin designed to optimize patient positioning, privacy, and imaging conditions and wherein said system is deployable in distributed care settings including MedSpas, pharmacies, imaging centers, and primary care clinics.

Embodiments of the disclosed invention have been referenced in detail, and one or more examples of the disclosed invention have also been illustrated in the accompanying figures. Each of the embodiments and examples herein have been provided to explain the present technology, not as limitations of the present technology. Furthermore, while particular embodiments of the invention have been described in detail, it will be appreciated that alterations to, variations of, and equivalents to these embodiments may be readily conceived of by those skilled in the art, upon attaining an understanding of the foregoing. For instance, features illustrated or described with respect to one embodiment may be used with another embodiment to yield an additional embodiment. It is intended that the present subject matter covers all such modifications and variations within the scope of the appended claims and their equivalents. Those of ordinary skill in the art may practice these and other modifications and variations to the present invention without departing from the scope of the present invention, which is more particularly set forth in the appended claims. Furthermore, the foregoing description is by way of example only, and is not intended to limit the invention, as will be appreciated by those of ordinary skill in the art.

1. A system for autonomous dermoscopic skin imaging and lesion risk assessment comprising:
a guidance module comprising one or more three-dimensional (3D) imaging sensors configured to detect locations of one or more regions of skin of a patient, and a communication device configured to communicate one or more prompts to direct positioning of the patient before or during image acquisition;
a plurality of high-resolution optical sensors configured to capture images of the one or more regions of the skin of the patient; and
a processor communicatively coupled to a memory, the 3D imaging sensors, the communication device, and the plurality of high-resolution optical sensors, wherein the processor is configured to read instructions stored in the memory which cause the processor to:
a) trigger one or more of the 3D imaging sensors to detect a position of the patient;
b) analyze the detected position of the patient by comparing it to a reference position;
c) when the detected position matches the reference position within a tolerance, trigger one or more of the plurality of high-resolution optical sensors to capture the images of the one or more regions of the skin of the patient;
d) when the detected position does not match the reference position within the tolerance, trigger the communication device to provide the one or more prompts to the patient to change their position and repeat operations a) through d);
e) process the captured images to generate a 3D digital model of the skin of the
f) generate a representation of the 3D digital model with metadata and associated malignancy risk scores for one or more objects in the 3D digital model, wherein the one or more objects are classified using one or more artificial intelligence (AI) models.

2. The system of claim 1, further comprising a display communicatively coupled to the processor, wherein the display is configured to display the representation of the 3D digital model with the metadata and the associated malignancy risk scores for the one or more objects.

3. The system of claim 1, wherein the metadata comprises one or more of: location data or size data for the one or more objects.

4. (canceled)

5. The system of claim 1, wherein the one or more 3D imaging sensors comprise time-of-flight (ToF) sensors, stereoscopic cameras, or other laser-based distance measurement sensors.

6. The system of claim 1, wherein the communication device comprises one or both of: an audio speaker and a visual display.

7. The system of claim 1, further comprising:
a plurality of illumination sources;
a first polarization filter configured to be positioned between an illumination source of the plurality of illumination sources and a region of skin of the one or more regions of the skin of the patient; and
a second polarization filter configured to be positioned between the region of skin of the one or more regions of the skin of the patient and a high-resolution optical sensor of the plurality of high-resolution optical sensors;
wherein a second polarization axis of the second polarization filter is approximately perpendicular to a first polarization axis of the first polarization filter.

8. The system of claim 1, wherein the processing the images to generate a 3D digital model of the skin of the patient further comprises cropping out regions of one or more of the captured images and combining the captured images taken at the one or more regions of the skin of the patient together into a composite image.

9. The system of claim 1, wherein the processing the images to generate a 3D digital model of the skin of the patient further comprises forming a 3D point cloud using information from the one or more 3D imaging sensors, and positioning the captured images in 3D space to generate the 3D digital model using projection mapping or depth-image fusion.

10. The system of claim 1, wherein the images each comprise a resolution from about 10 microns to about 200 microns, or less than about 25 microns.

11. A computer-implemented method for autonomous dermoscopic skin imaging and lesion risk assessment, comprising:
a) instructing one or more three-dimensional (3D) imaging sensors to measure locations of one or more regions of skin of a patient;
b) detecting a position of the patient using measured locations of one or more regions of skin of a patient;
c) analyzing the detected position of the patient, wherein the detected position of the patient is compared to a reference position;
d) if the detected position matches the reference position within a tolerance, the computer-implemented method proceeds to operation e), and if the detected position does not match the reference position within the tolerance, then the computer-implemented method proceeds by instructing a communication device to provide visual and/or audio prompts to the patient to change their position and then repeating operations a) through d);
e) instructing a plurality of high-resolution optical sensors to capture images of the one or more regions of the skin of the patient; and
f) processing the images to generate a 3D digital model of the skin; and
g) generating a representation of the 3D digital model with metadata and associated malignancy risk scores for one or more objects in the 3D digital model, wherein the one or more objects are classified using one or more artificial intelligence (AI) models.

12. (canceled)

13. The computer-implemented method of claim 11, wherein the images each comprise a resolution from about 10 microns to about 200 microns, or less about 25 microns.

14. A method of controlling an autonomous dermoscopic skin imaging and lesion risk assessment system, comprising:
a) measuring locations of one or more regions of skin of a patient using one or more three-dimensional (3D) imaging sensors coupled to a computing system of the autonomous dermoscopic skin imaging and lesion risk assessment system, wherein the computing system comprises a processor and memory;
b) detecting a position of the patient using the one or more of the 3D imaging sensors;
c) analyzing the detected position of the patient using the computing system, wherein the detected position of the patient is compared to a reference position stored in the memory of the computing system;
d) if the detected position matches the reference position within a tolerance, the method proceeds to operation e), and if the detected position does not match the reference position within the tolerance, then the method proceeds by instructing a communication device to provide visual and/or audio prompts to the patient to change their position and then repeating operations a) through d);
e) capturing images of the one or more regions of the of the patient using a plurality of high-resolution optical sensors of the autonomous dermoscopic skin imaging and lesion risk assessment system;
f) processing the images to generate a 3D digital model of the skin using the computing system; and
g) generating a representation of the 3D digital model with metadata and associated malignancy risk scores for one or more skin lesions using the computing system, wherein the one or more skin lesions are classified using one or more artificial intelligence (AI) modules of the computing system.

15. The method of claim 14, wherein the high-resolution optical sensors are each from about 30 cm to about 60 cm away from the skin when capturing the images.

16. The method of claim 14, wherein the images of the skin are captured over greater than about 50% or greater than about 90% of the skin of the patient.

17. The method of claim 14, wherein the images each comprise a resolution from about 10 microns to about 200 microns, or less than about 25 microns.

18. The method of claim 14, wherein the 3D imaging sensors comprise time-of-flight (ToF) sensors, stereoscopic cameras, or other laser-based distance measurement sensors.

19. The method of claim 14, further comprising generating a recommendation for a treatment of the one or more skin lesions using the computing system comprising recommending one or more of: further observation and monitoring, application of topical medicated creams or ointments, cryotherapy, surgical excision, laser therapy, photodynamic therapy, chemotherapy, radiation therapy, targeted drug therapy, and immunotherapy.

20. The method of claim 14, further comprising treating a skin lesion of the one or more skin lesions using a treatment comprising one or more of: further observation and monitoring, application of topical medicated creams or ointments, cryotherapy, surgical excision, laser therapy, photodynamic therapy, chemotherapy, radiation therapy, targeted drug therapy, and immunotherapy.

21. The system of claim 1, wherein the processor is further configured to read instructions stored in the memory which cause the processor to:
segment the one or more objects in the 3D digital model using a segmentation AI model of the one or more AI models;
classify the segmented objects into one or more categories using a classification AI model of the one or more AI models, wherein the classification AI model is trained on biopsy-confirmed dermoscopic datasets, and one of the one or more categories comprises skin lesions; and
assign the malignancy risk scores to the one or more of the classified segmented objects using the classification AI model.

22. The computer-implemented method of claim 11, further comprising:
segmenting the one or more objects in the 3D digital model using a segmentation AI model of the one or more AI models;
classifying the segmented objects into categories using a classification AI model of the one or more AI models, wherein the classification AI model is trained on biopsy-confirmed dermoscopic datasets, wherein one of the categories comprises skin lesions; and
assigning the malignancy risk scores to the one or more of the classified segmented objects using the classification AI model.

23. The method of claim 14, further comprising:
segmenting and classifying the one or more regions of the skin as one or more skin lesions in the 3D digital model using an AI module of the one or more AI modules, wherein the AI module is trained on biopsy-confirmed dermoscopic datasets; and
assigning the malignancy risk scores to each identified skin lesion using the AI module.
